(19) United States
(12) Patent Application Publication
(10) Pub. No.: US 2024/0019438 A1
(43) Pub. Date: Jan. 18, 2024

(54) ASSAYS FOR RAPID DETECTION OF AIRBORNE VIRUSES INCLUDING INFLUENZA AND CORONAVIRUSES

(71) Applicant: TELEDYNE FLIR DETECTION, INC., Stillwater, OK (US)

(72) Inventors: Jeremy P. Walker, Stillwater, OK (US); Jennifer L. POOLE, Stillwater, OK (US); Kristin KORTE, Stillwater, OK (US)

(21) Appl. No.: 18/256,844

(22) PCT Filed: Dec. 31, 2021
(86) PCT No.: PCT/US2021/073208

§ 371 Date: Jun. 9, 2023

Related U.S. Application Data
(60) Provisional application No. 63/133,209, filed on Dec. 31, 2020.

(51) Int. Cl. G01N 33/569

(52) U.S. Cl. CPC G01N 33/56983; G01N 2333/4724; G01N 2333/165; G01N 2333/11; G01N 2333/115; G01N 2333/405

(57) ABSTRACT

Disclosed are compositions that comprise one or more broad-spectrum capture molecules (including, for example, the small, homodimer-forming lectin protein, Griffithsin), or glycoproteins that coat the viral envelope surface in methods for the identification of one or more virus particles in an airborne, aerosol, or aerosolized sample. Also disclosed are methods for the use of such capture agents in the manufacture of diagnostic reagents (as well as kits, devices, and systems comprising them), useful in developing viral detection platforms that are both rapid and facile to perform, yet highly-sophisticated, accurate, and sensitive. Methods are also provided for using these compositions in the identification, molecular capture, characterization, and design of therapeutic regents related thereto for the treatment of one or more symptoms of a viral infection, or a virally-induced disease in mammals and, particularly, in humans.

### BACKGROUND

### Technical Field

The present disclosure relates generally to environmental medical methods, and in particular, to near real-time diagnostic assays for detecting and/or quantitating one or more airborne environmental pathogens, and in particular, viruses such as influenza, coronaviruses, and other agents of human disease.

Compositions and/or methods are provided, which are useful in the rapid detection of one or more Influenza viruses, one or more severe acute respiratory syndrome (SARS) coronavirus (CoV) (and particularly, one or more SARS CoV-2, the causal agent of COVID-19) or combinations thereof in environmental, and particularly, airborne, samples. In particular applications, near real-time detection of such pathogens is possible using continuous bio-monitor devices.

### Description of Related Art

Coronaviruses

Coronaviruses are a group of related RNA viruses that cause diseases in mammals and birds. In humans and birds, they cause respiratory tract infections that can range from mild to lethal. Mild illnesses in humans include some cases of the common cold (which is also caused by other viruses, predominantly rhinoviruses), while the three more lethal varieties cause SARS, MERS, and COVID-19, respectively. The viruses cause gastroenteritis and/or diarrhea in bovines and porcines, while hepatitis and encephalomyelitis are the principal illnesses observed in murine infections.

Coronaviruses constitute the subfamily Orthocoronavirinae, in the family Coronaviridae, order Nidovirales. They are enveloped viruses with a positive-sense single-stranded RNA genome and a helically symmetric nucleocapsid. The genome size of most coronaviruses ranges from approximately 26 to 32 kilobases, which is one of the largest among RNA viruses. They have characteristic club-shaped spikes that project from their surface, which in electron micrographs create an image reminiscent of the solar corona, from which their name derives. Coronaviruses are named for their appearance: Under the microscope, the viruses look like they are covered with pointed structures that surround them like a corona, or crown.

Diseases of Coronaviral Origin

Four human coronaviruses produce symptoms that are generally mild: 1) Human coronavirus OC43 (HCoV-OC43); β-CoV; 2) Human coronavirus HKU1 (HCoV-HKU1), β-CoV; 3) Human coronavirus 229E (HCoV-229E), α-CoV; and 4) Human coronavirus NL63 (HCoV-NL63), α-CoV.

Infected carriers are able to shed viruses into the environment. The interaction of the coronavirus spike protein with its complementary cell receptor is central in determining the tissue tropism, infectivity, and species range of the released virus. Coronaviruses mainly target epithelial cells. They are transmitted from one host to another host, depending on the coronavirus species, by either an aerosol, fomite, or fecal-oral route.

Human coronaviruses infect the epithelial cells of the respiratory tract, while animal coronaviruses generally infect the epithelial cells of the digestive tract. SARS coronavirus, for example, infects via an aerosol route, the human epithelial cells of the lungs by binding to the angiotensin-converting enzyme 2 (ACE2) receptor. Transmissible gastroenteritis coronavirus (TGEV) infects, via a fecal-oral route, the pig epithelial cells of the digestive tract by binding to the alanine aminopeptidase (APN) receptor.

Severe Acute Respiratory Syndrome (SARS)

Severe acute respiratory syndrome (hereinafter abbreviated “SARS”) is a viral respiratory disease of zoonotic origin caused by a SARS-associated coronavirus (initially “SARS-CoV,” now “SARS-CoV-1”). SARS was first identified in Guangdong, CHINA, a coastal province on the north shore of the South China Sea in early November 2002. Between mid-November 2002 and Jul. 1, 2003, a total of just over 8,000 cases of the infection were reported in 29 countries, with a majority of cases being reported in China, Hong Kong, Taiwan, Canada, Singapore, and South Vietnam. According to the World Health Organization (WHO), the mortality for patients afflicted with SARS was 11% on average, and approximately 50% in patients aged 65 and over. A total of 774 deaths were attributed to the disease, with the last case being reported nine months since initial outbreak.

Major clinical symptoms of SARS include a fever with temperatures of 38° C. or higher and respiratory problems, such as coughing and difficulty of breathing. In some cases, symptoms such as headache, shaking chills, loss of appetite, generalized malaise, diarrhea, or clouding of consciousness are observed. However, these symptoms are almost the same as those of other respiratory diseases, such as influenza. Thus, it is difficult to distinguish SARS from other diseases based solely on its symptoms. As of 2020, no specific vaccine or treatment for the disease is available.

Middle East Respiratory Syndrome (MERS)

Middle East Respiratory Syndrome (hereinafter abbreviated “MERS” and also known as “camel flu”) is a viral respiratory infection caused by the MERS-coronavirus (MERS-CoV). The first case of MERS was identified in Jeddah, SAUDI ARABIA in June 2012, and by early 2020, a total of 2500 cases had been reported, primarily on the Arabian Peninsula. Less than 1000 fatalities due to MERS have been reported.

Symptoms of MERS include fever, coughing, shortness of breath, headache, malaise, and gastrointestinal discomfort. Cause of the disease was linked to infection with MERS coronavirus, (“MERS-CoV” or “EMC/2012”), a single-strand RNA virus belonging to the genus Betacoronavirus which is genetically distinct from both the SARS coronavirus and the common-cold coronavirus. As of 2020, no specific vaccine or treatment for the disease is available, although extra-corporeal membrane oxygenation appears to improve outcomes significantly.

Coronavirus Disease 2019 (COVID-19)

Signs of another related infectious disease, which became known as COVID-19, first appeared in Wuhan, CHINA in December 2019; and while WHO officials are still tracing the exact source of this latest coronavirus outbreak, early hypotheses thought it may be linked to a seafood market, where several visitors developed symptoms of a viral pneumonia, which was later linked to a novel coronavirus, designated SARS-CoV-2.

A study published on Jan. 25, 2020 noted that the individual with the first reported case of COVID-19 became ill on Dec. 1, 2019 but had no link to the seafood market. Epidemiological investigations are ongoing as to how this virus originated and spread. As of Dec. 31, 2020, more than 1,811,000 deaths have been attributed to CoV-2 infection worldwide, while more than 83 million people have been sickened by the illness. More than 345,000 of those were reported in the United States alone. As of the filing date of this application, no specific treatment has been available publicly, although several commercial vaccines have completed clinical trials, and are now being distributed in a limited number of countries—primarily to medical personnel, first responders, and at-risk populations.

COVID-19 has served as a stark reminder of how prolific the spread of airborne infectious disease can be. Viremic windows and incubation periods of this disease vary to extremes such that people are capable of competently spreading illness long before symptoms appear, if they ever do. This leaves a critical gap of unknown certainties and risks society is left to navigate as society seeks to return to some form of normalcy attending school, work, and leisure activities. While sensors are available today to detect atmospheric threats in real time, automated air sampling, characterization, and identification of what anomalies are found in air, are not.

The SARS-CoV-2 virus contains approximately 65 surface spike proteins that bind with very high affinity to the ACE-2 enzyme. Numerous ACE-2 enzymes can bind to a single virus (first amplification), and delivery of a substrate that becomes fluorescent when catalytically cleaved by ACE-2 (second amplification) generates a measurable signal that triggers an alarm in the device. The orthogonal triggering and identification provides a robust and rapid capability to sensitively detect SARS-CoV-2 in public spaces.

Diagnostic Assays for SARS Coronavirus

An immunologic procedure has been known as a method of clinical testing. In such testing, the presence of an antibody against a viral antigen in blood, serum, urine, or saliva is inspected. The enzyme-linked immunosorbent assay (ELISA) and the immunofluorescence assay (IFA) are known techniques for detecting antibodies against the SARS coronavirus. With these techniques, however, antibodies cannot be detected at the early stage of the disease. In the case of ELISA, antibodies cannot be detected until 20 days after the development of the disease. In the case of IFA, antibodies cannot be detected until 10 days after the development of the disease.

Also, a method for detecting antibodies via amplification of the virus gene via PCR has been known. This technique, however, has been problematic since it takes 1 hour or longer for amplification and detection, and the detection sensitivity thereof is low. Accordingly, a method for detecting the SARS coronavirus with rapidity and high sensitivity is sorely lacking).

### Deficiencies in the Prior Art

Presently the state of the art in biological identification is either the use of lateral flow assays (LFAs) or polymerase chain reaction (PCR), which in either case require collection and retrieval of a viral sample, coupled with some level of purification. LFAs utilize antibody chemistry to move a bound gold colloidal particle along a test strip, similar to a home pregnancy test. PCR requires rupture of the viral membrane to access the internal RNA, which is the subjected to amplification to identify the sequence against a library in order to identify the pathogen. Neither of these techniques are adaptable to automated, real-time use.

### SUMMARY OF THE DISCLOSURE

The present invention overcomes these, and other limitations inherent in the prior art by providing compositions for detecting one or more viral compositions in a biological sample.

It is an object of the present invention to detect a pathogenic virus, (e.g., an influenza virus, or a coronavirus such as a SARS-CoV-1, the causal agent of SARS, MERS-CoV, or SARS-CoV-2, the causal agents of Middle East Respiratory Syndrome and COVID-19, respectively; with high sensitivity for early detection and/or diagnosis of a disease process in a mammalian subject infected with one or more such viral isolates.

In an important aspect, the invention provides a novel, highly-specific enzyme-based assay that utilizes a dual-amplification strategy to catalytically report the detection of viruses such as the SARS-CoV-2 virus within an already proven light-scattering trigger device, the IBAC (Instantaneous Biological Aerosol Counter) duo. The sequential triggering function of the IBAC when aerosolized particles are detected will prompt collection and concentration of viruses onto a stationary phase that is then interrogated with the double-amplification, affinity-based enzymatic reporter assay. Efficient capture and concentration of viruses from the moving airstream will leverage versatile lectin-glycoprotein affinity chemistry. Delivery of reagents that specifically target the SARS-CoV-2 virus via the human angiotensin converting Enzyme-2 (ACE-2) binding, will enable sensitive detection.

In certain embodiments, the disclosed innovation concern continuous enzymatic assays for the capture of viruses in an airstream, coupled with addition of enzymatic and fluorogenic reagents capable of positively identifying the presence of either influenza or coronaviruses in the air.

A key innovation herein is the use of a lectin-based capture motif—in particular, a griffithsin polypeptide or fragment thereof, that very specifically targets virus envelop glycoproteins and binds them. Lectins are small proteins that bind with high affinity to viral envelop glycoproteins. A comprehensive review of lectins useful in the practice of the present disclosure is found in Slifkin and Doyle (1990) and Dan et al. (2016). Griffithsin protein herein is used as the capture molecule immobilized on an impactor disk that ‘grabs’ the virus out of the moving airstream as it impacts the surface. The ability of lectin-based peptides to capture and concentrate viral particles from a moving airstream represents an important feature of the disclosed embodiment. Class-type capture is applicable even toward future unknown viral threats (for example, as yet uncharacterized CoV or Influenza viruses that are the etiological agents of other mammalian viral diseases).

Once immobilized, the viruses can then be assayed for surface enzyme activity. The device may be used to detect viruses in exhaled breath in airports, hospitals, security checkpoints, as well as in any area where large crowds of people may present a pathogen transmission risk. Because CoV are known to bind to the cell surface enzyme, ACE-2, during infection, and numerous ACE-2 enzyme molecules can bind to a single viral particle, a fluorescent substrate can be exploited (i.e., catalytically turned over) to amplify viral detection into a measurable signal. The use of ACE-2 enzyme as a reporter molecule provides a double-amplification capability that enables 4- to 5-log amplification. Use of this natural target makes the assay less susceptible to interruption due to mutations in viral RNA/proteins, etc.

Importantly, because lectin capture and ACE-2 interrogation are non-destructive techniques, the viral samples may be retained intact during assay, for enabling subsequent “off-device” validation of data with one or more additional assay techniques, including, for example, PCR. Samples can be cross referenced with existing filter collection in the IBAC-2 sampling unit if desired.

In one aspect, a method for detecting a virus is provided. The method comprises contacting an air sample including virus particles with a binding agent on a support, thereby binding at least a portion of the virus particles in the air sample to the binding agent on the support. The virus particles bound to the binding agent on the support are contacted with a reporter that binds specifically to and/or is cleaved specifically by a surface protein of the virus particles or a glycoprotein of the virus particles. The presence of the reporter bound to and/or cleaved by the surface protein of the virus particles or the glycoprotein of the virus particles is detected.

In another aspect, a system for detecting the presence of virus particles is provided. The system comprises a support, an air pump, a fluid delivery apparatus, a sensor, and a programmable hardware device. The support comprises a binding agent configured to bind at least a portion of virus particles in an air sample. The air pump is configured to deliver the air sample to the support. The fluid delivery apparatus is configured to deliver a reporter to the support. The reporter is configured to bind specifically to and/or to be cleaved specifically by a surface protein of the virus particles or a glycoprotein of the virus particles. The programmable hardware device is configured to measure a property of the support utilizing the sensor to thereby determine presence of the reporter.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following drawings form part of the present specification and are included to demonstrate certain aspects of the present invention. For promoting an understanding of the principles of the invention, reference will now be made to the embodiments, or examples, illustrated in the drawings and specific language will be used to describe the same. It will, nevertheless be understood that no limitation of the scope of the invention is thereby intended. Any alterations and further modifications in the described embodiments, and any further applications of the principles of the invention as described herein are contemplated as would normally occur to one of ordinary skill in the art to which the invention relates.

The invention may be better understood by reference to the following description taken in conjunction with the accompanying drawings, in which like reference numerals identify like elements, and in which:

FIG. 1 illustrates the frequency with which new diseases are continuously emerging. The current paradigm that we have towards viral detection is to search for only the virus of interest in a sample using specific tools like antibodies and PCR. When new viruses emerge, reliance on these specific techniques slows the detection and study of these entities because new tools must be generated to fit the current research and detection paradigms. PCR primers can take weeks-to-months to generate and good antibodies can take 6+ months or more. The present methods provide a test for general virus detection that can be later modified to include strain specificity as new information and tools emerge;

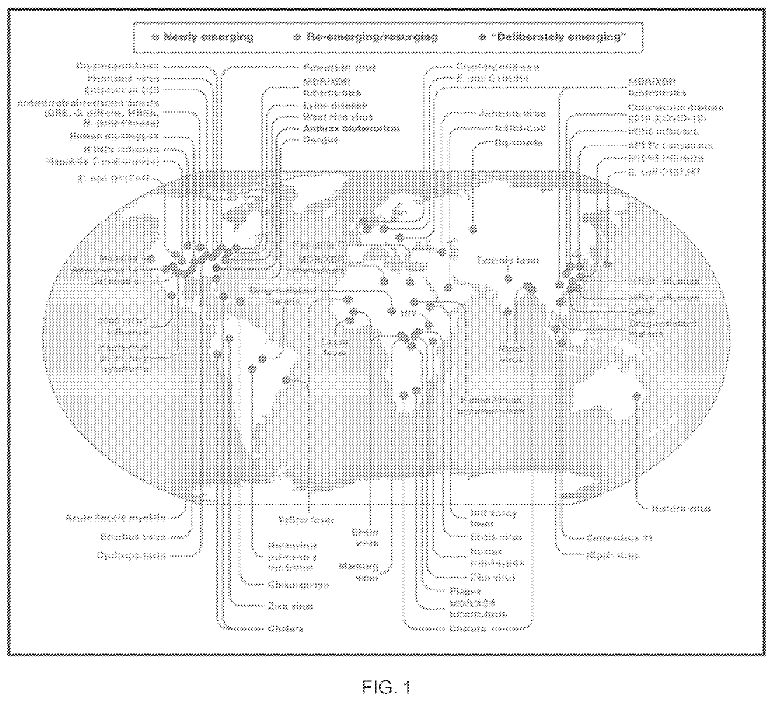

FIG. 2A and FIG. 2B show that most contemporary virus testing rely either on the use of PCR or antibody-based detection assays; the referenced article provides background;

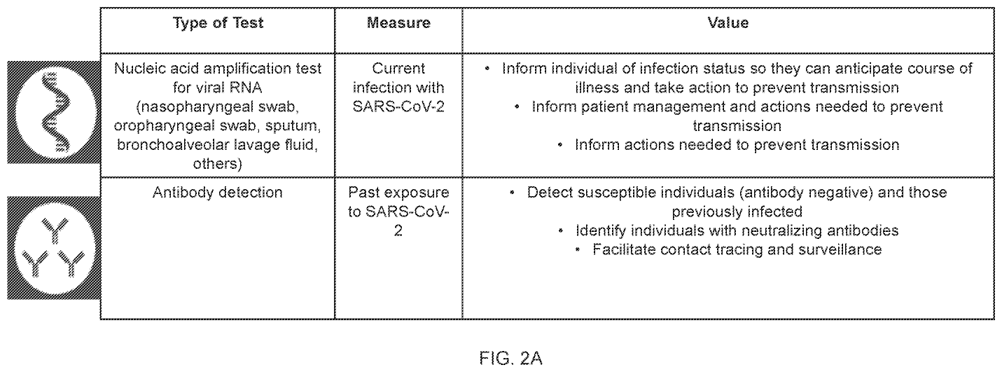

FIG. 3 show application of the “general virus capture and detection” technology to be particularly advantageous in environmental monitoring (such as air, water, and/or surface monitoring) and diagnostics (such as use of the final assay for virus detection and/or use of the capture technology to enhance capture consumables associated with current assays to improve sensitivity); an additional embodiment of the disclosure is the use of these interactions not for detection, but for enhancing PPE properties;

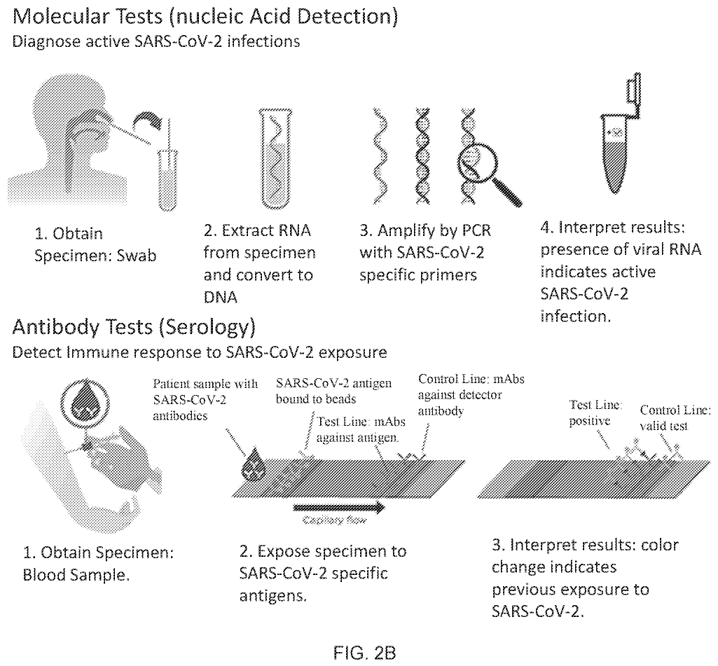

FIG. 4 shows SARS-CoV-2 Assay: 1) Griffithsin-functionalize impactor plate 2) capture of virus in air stream; 3) addition of ACE2 enzyme that binds to virus; 4) rinse to remove unbound ACE2; and 5) addition of substrate and generation of fluorescent signal and corresponding optical fluorescence signal. In the most generic sense, the viral assay involves an Enzyme-Linked Lectin Assay (ELLA); the difference that binding to *any* virus is preferable, whereas in the past, such assays have preferentially targeted *specific* viruses. Targeting of *any* virus may require the use of either a single lectin or a mixture of lectins to get broad applicability; viral samples are typically provided in the form of liquid or air introduced to the immobilized capture lectin. Once bound, sampling plates are washed free of unbound material then treated with a secondary reagent of enzyme-linked lectin. Enzyme linked lectin may employ traditional ELISA reagents such as horseradish peroxidase (HRP), etc., while unbound enzyme-linked lectin is washed away, and enzyme substrate is then added to produce a signal; enzyme-linked lectin assays (ELLA) have been described generically in previous publications (McCoy et al., 1984; Gao et al., 2016; Couzens et al., 2014; Prevato et al., 2015; and Suda et al., 2015);

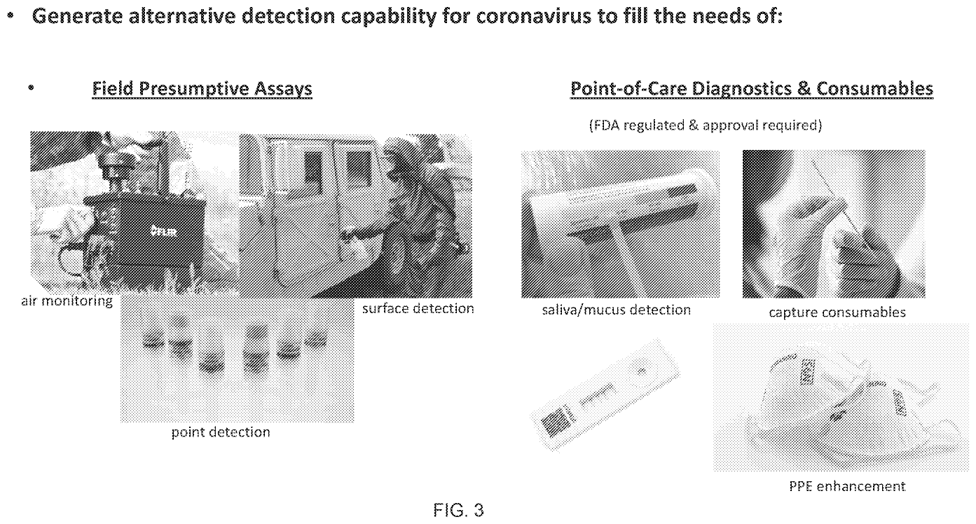

FIG. 5A summarizes a variety of detectable pathogens, and the disease(s) associated with each pathogen; FIG. 5B summarizes the lectins, a group of thermostable, carbohydrate-binding proteins (CBP) that are highly specific for recognition of sugar groups lectin binding;

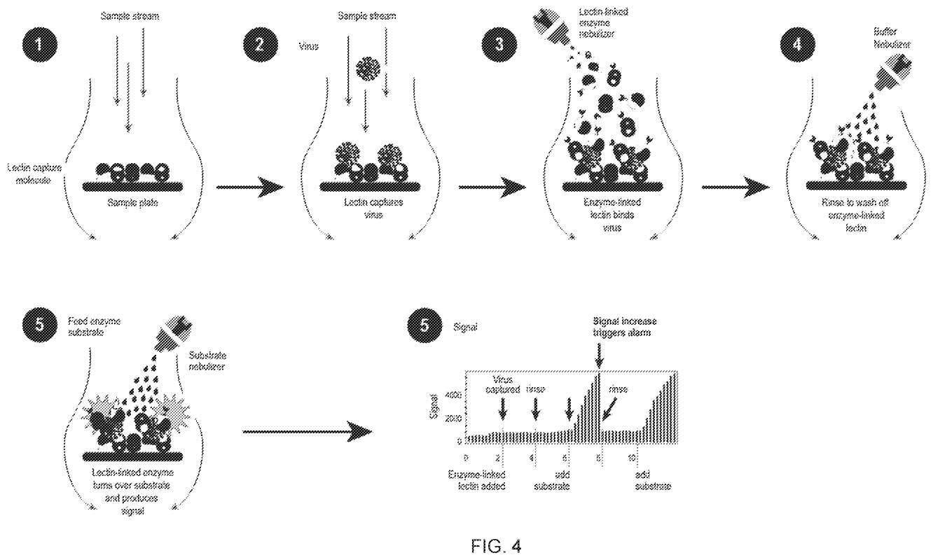

FIG. 6A and FIG. 6B summarize the virus-specific detection using the lectin-binding motif to capture glycoproteins on viruses, additional specificity in virus identification can be generated based on the particular structure and interactions of individual viruses; FIG. 6A shows the glycosylation profile of viruses is somewhat dependent on the cells in which they are made (species, and location within organism), but most viruses tend to have high-mannose glycosylation. Using (one or a few different) lectins that target this high-mannose feature, a capture scheme can be generated to bind generically to any virus in the system. Since there are not *good viruses* in systems like there are *good bacteria*, it would be of general interest to know if ANY virus were present; In addition to being more generalizable than other capture methods, such as antibodies, lectins also tend to be much easier to manufacture (because of their smaller size) and they have been shown to be much more stable (more resistant to heat means less need for cold chain storage for any developed product); For example, the Neuraminidase of influenza virus can be used as a reporter once virus is bound to immobilized lectin. Coronaviruses (including SARS-Cov-2) uses spike glycoproteins on the virus surface to bind human ACE2 receptors on the surface of cells. This interaction with ACE2 can be used as a reporter once virus is bound to immobilized lectin (FIG. 6B);

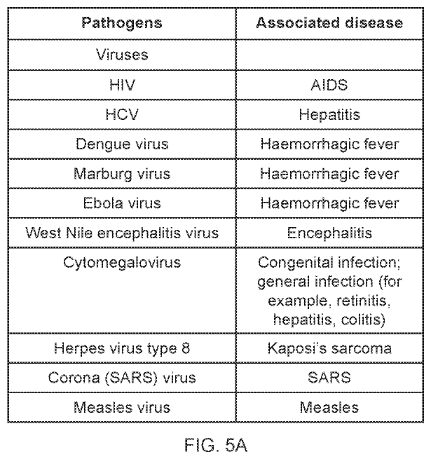

FIG. 7 shows an exemplary lectin influenza assay in accordance with one aspect of the present disclosure. As more information about a specific virus is known, the lectin assay can be used to conduct more specific detection of viruses utilizing enzymes that are present within/or bind to the virus of interest. For example, detection of the coronavirus is known to interact with the ACE2 enzyme on human cells. By probing lectin-bound virus with ACE2 instead of enzyme-linked lectin, specific detection of the coronavirus class of viruses is obtained; virus sample would be in the form of liquid or air introduced to the immobilized capture lectin. Once bound, the sampling plate would be washed free of unbound material then treated with a secondary reagent of ACE2 enzyme. Unbound ACE2 would be washed away, and enzyme substrate would then be added to produce a signal. A similar assay has been conducted using lectin to detect influenza virus hemagglutinin in a sample via immunoassay (PCT Intl. Pat. Appl. Publ. No. WO 2013/088367, the entire contents of which is specifically incorporated herein in its entirety by express reference thereto); also, oligosaccharides to capture influenza virus to enhance virus concentration for PCR assay from saliva samples (Suda et al., 2015);

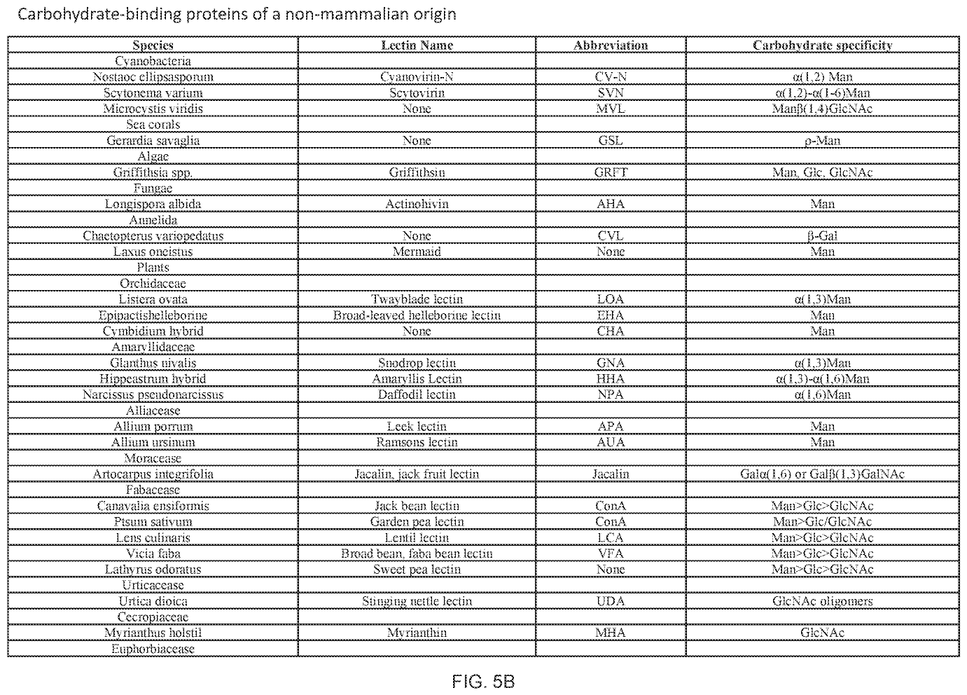

FIG. 8 shows an exemplary lectin influenza assay in accordance with one aspect of the present disclosure. Once more information about a specific virus is known, then the lectin assay can be used to conduct more specific detection of viruses utilizing enzymes that are present within/or bind to the virus of interest; for example, detection of the coronavirus is known to interact with the ACE2 enzyme on human cells. By probing lectin-bound virus with ACE2 instead of enzyme-linked lectin, specific detection of the coronavirus class of viruses is obtained; Virus sample would be in the form of liquid or air introduced to the immobilized capture lectin. Once bound, the sampling plate would be washed free of unbound material then treated with a secondary reagent of ACE2 enzyme. Unbound ACE2 would be washed away, and enzyme substrate would then be added to produce a signal;

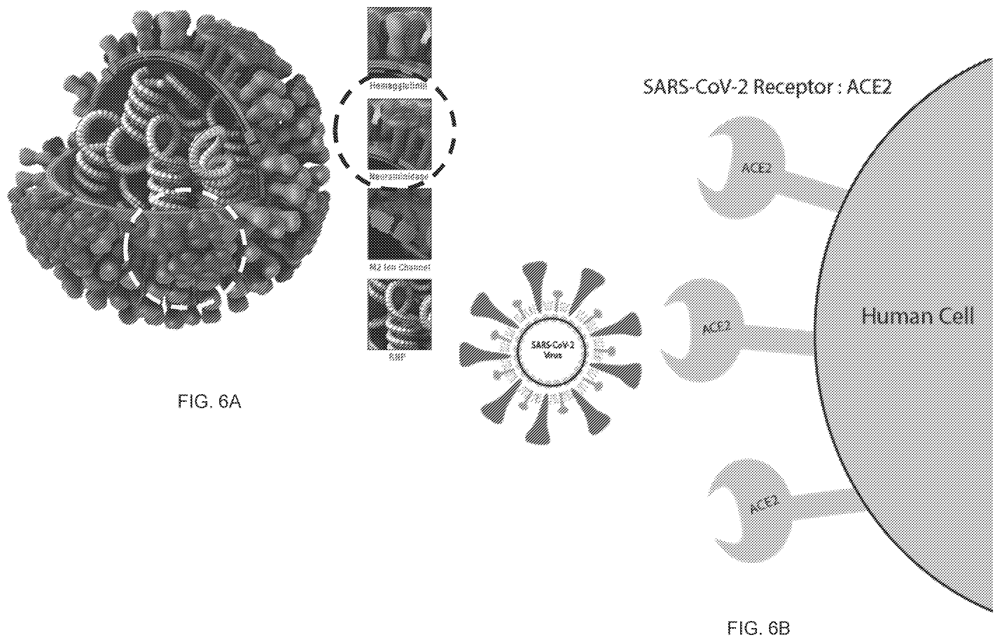

FIG. 9A and FIG. 9B show an exemplary detection agent and the results of certain coronavirus assays in accordance with particular aspects of the present disclosure; FIG. 9C shows the results of a spike assay in accordance with one aspect of the present invention;

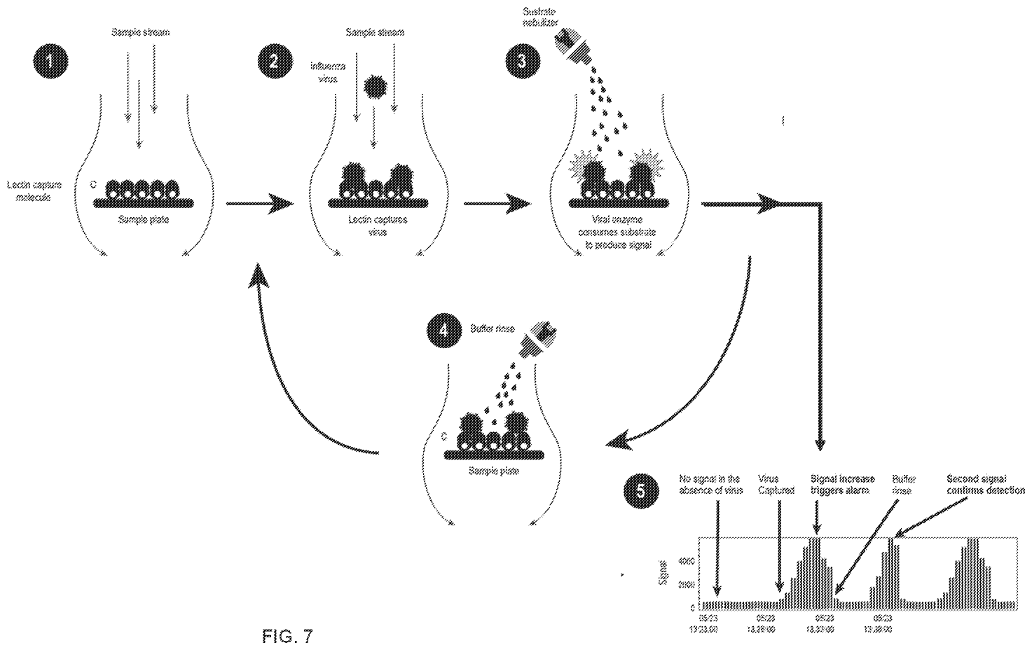

FIG. 10A and FIG. 10B show an exemplary influenza detection consideration in accordance with one aspect of the present disclosure;

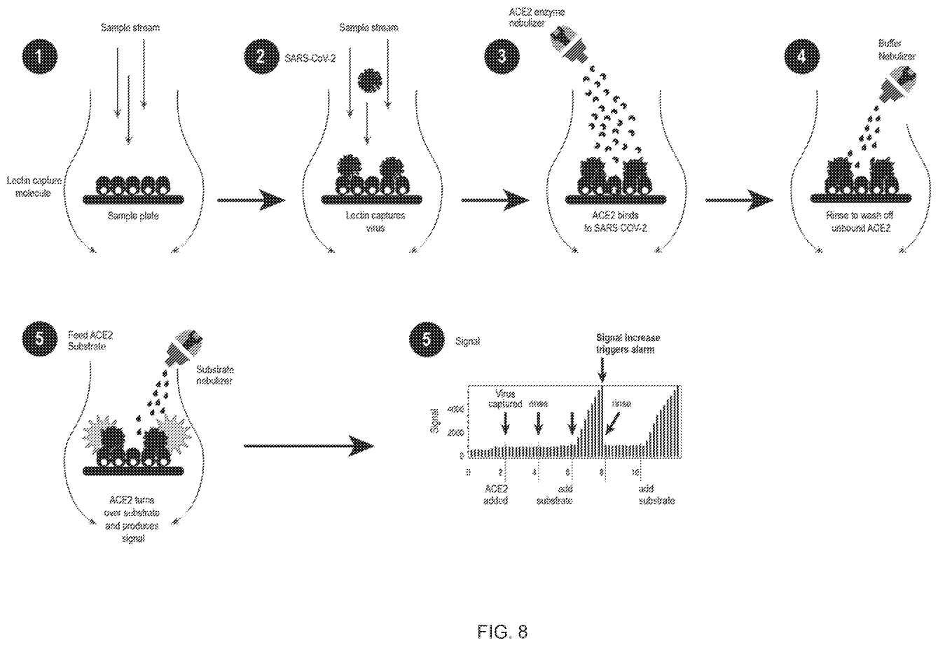

FIG. 11A, FIG. 11B, FIG. 11C, and FIG. 11D show an exemplary coronavirus detection consideration in accordance with one aspect of the present disclosure;

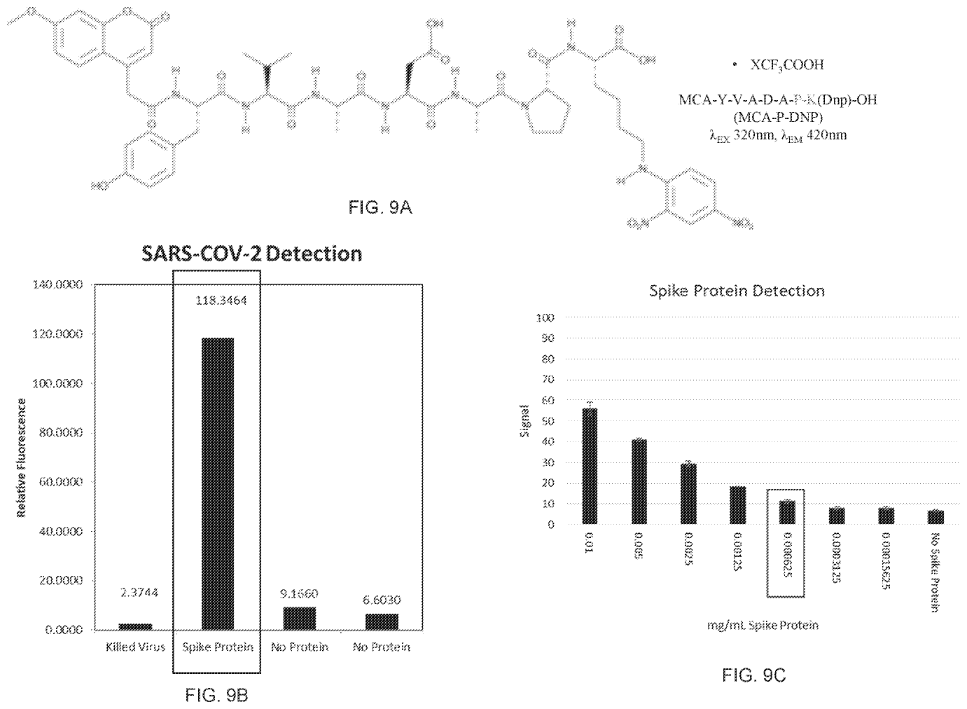

FIG. 12 is an illustration showing exemplary CoV-2, Influenza, and rhinovirus structures and approximate mean diameters, in accordance with one aspect of the present disclosure;

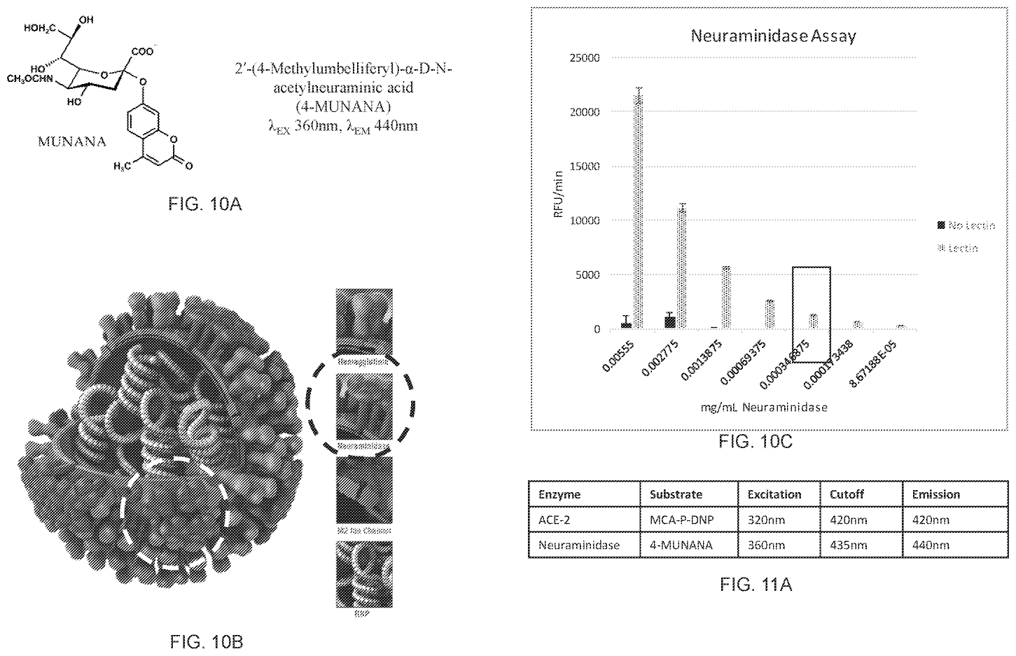

FIG. 13 shows the Instantaneous Biological Aerosol Counter-2 (IBAC-2) triggers and alarms when pathogen aerosol is detected;

FIG. 14A and FIG. 14B show illustrative examples of IBAC hardware including internal plumbing, fluid consumables, and software GUI. FIG. 14B shows the raw signal (top) shows the periodic amplitude of the assay and the impact of the presence of nerve agent. The other graphs illustrate the rolling amplitude and amplitude change that generate the alarm status (bottom);

FIG. 15 shows ACE2 bound to SARS-CoV-2 at the S-protein (red) will turn over the fluorogenic polypeptide substrate by cleaving the Pro-Lys bond to produce a fluorescent signal at 381 nm. Appearance of fluorescence (violet) indicates SARS-CoV-2 is bound; and

FIG. 16 shows the surface neuraminidase enzyme (red) on influenza cleaves the MUNANA substrate and produces a fluorescent 4-MU product that emits at 441 nm.

FIG. 17 illustrates a non-limiting embodiment of a method for detecting virus particles according to the present disclosure; and

FIG. 18 illustrates a schematic of a non-limiting embodiment of a system for detecting virus particles according to the present disclosure.

### BRIEF DESCRIPTION OF THE NUCLEOTIDE AND POLYPEPTIDE SEQUENCES

SEQ ID NO:1 is the amino acid sequence of algal Griffithsin, in accordance with one aspect of the present disclosure:

SLTHRKFGGSGGSPFSGLSSIAVRSGSYLDXIIIDGVHHGGSGGNLSPTF
TFGSGEYISNMTIRSGDYIDNISFETNMGRRFGPYGGSGGSANTLSNVKV
IQINGSAGDYLDSLDIYYEQY

### DESCRIPTION OF ILLUSTRATIVE EMBODIMENTS

Illustrative embodiments of the invention are described below. In the interest of clarity, not all features of an actual implementation are described in this specification. It will of course be appreciated that in the development of any such actual embodiment, numerous implementation-specific decisions must be made to achieve the developers' specific goals, such as compliance with system-related and business-related constraints, which will vary from one implementation to another. Moreover, it will be appreciated that such a development effort might be complex and time-consuming but would be a routine undertaking for those of ordinary skill in the art having the benefit of this disclosure.

The airborne spread of pathogens poses a significant risk to US warfighters, as well as the US civilian population, as illustrated by the COVID-19 pandemic. The SARS-CoV-2 virus has proven to be transmitted via aerosolized particles expelled from coughs, sneezes, and even talking and breathing. Further, the virus has been shown to be transmitted predominantly by asymptomatic individuals, creating a significant risk of spread in public places such as schools, transportation hubs, workplaces, and commercial spaces. The current inability to detect environmental pathogens in real time presents a substantial challenge. As a result, government officials have been forced to take a scattered approach in their recommendations toward monitoring and controlling virus transmission which have included techniques such as masking, thermal monitoring, sentinel diagnostic screening, contact tracing and enacting quarantines and restrictions on gathering size and travel. Each of these efforts can be useful; however, these techniques provide very little with respect to real-time actionable information and hinge primarily on preventative measures which require strict compliance in implementation to ensure efficacy. Even current diagnostic assays available for SARS-CoV-2 have a lag time of hours to days between test to result, and still require patients to consciously know that they have been exposed and therefore need to be tested. In the environmental monitoring field, optical methods alone are insufficient to discriminate classes of threats and truly characterize the causative agent of the anomaly detection. And, while the biochemical assays utilized in diagnostics exhibit the selectivity and sensitivity desired from an environmental monitor, the required sample manipulation, cycle-time and consumable burden have limited development and end-user adoption of these gold-standard techniques due to high logistics burdens and sustainment costs.

The development of the sensitive, real-time environmental aerosol monitor for viruses such as SARS-CoV-2 described herein provides access to a wealth of information previously unavailable to the study of pathogen transmission and illness progression. By successfully combining technologies could alert individuals during the act of exposure, which would allow for unprecedented data collection on exposure time and relative concentration of exposure in conjunction with better ascertaining the biomedical markers of SARS-CoV-2 infection and disease. Environmental monitoring techniques can be further optimized to facilitate situational tracing and contamination mitigation measures. The potential for environmental pathogen monitoring to identify the earliest spread of SARS-CoV-2 would have significant impact on subsequent infection rates, potentially improve the clinical outcomes and aid in preventing further economic or mission loss due to prolonged pandemic measures. Finally, the right approach will not only protect interior spaces from SARS-CoV-2, but serve to support both real-time microbiome monitoring and versatile backend identification approaches that can be rapidly expanded to accommodate Disease X.

This invention leverages unique surface protein properties and cellular entry mechanisms that viruses use to infect cells to develop highly specific assays that can be quantitated by measurement of fluorescent signal development. In one embodiment, key innovations of the disclosure include virus-targeted fluorescent enzymatic chemistries that can be deployed for continuous airborne detection in suitable devices, including, without limitation, the IBAC-Chem platform, or in a handheld test for exhaled breath for non-contact sampling and detection of the particular virus to be detected, including, for example one or more coronaviruses, such as a SARS-CoV-2 virus and/or one or more influenza viruses, such as Influenza A, Influenza B, Influenza C, Influenza D, and the like (including, but not limited to, Influenza A serotypes H1N1, H2N2, H3N2, H5N1, H7N7, H1N2, H9N2, N7N2, H7N3, H10N7, H10N7, H7N9, and H6N1). What is particularly attractive in such applications is the built-in amplification capability that comes from exploiting the distinctive surface enzyme chemistries associated with pathogen binding and quantification.

In one embodiment, there are disclosed methods explaining the highly-specific interaction between Griffithsin, a small, homodimer-forming lectin protein, and glycoproteins that coat the viral envelope surface. Griffithsin has been researched as an approach for blocking the viral binding mechanism leading to cellular entry; in vitro and in vivo prevention of HIV infection of human cells has been demonstrated. This binding affinity has been leveraged to enable viral capture from the incoming air sample. Griffithsin is immobilized on an impactor orthogonal to the direction of airflow. Incoming viral particles will be captured by the Griffithsin. Aqueous reagents nebulized into the air-stream will further react with bound viral particles and generate a fluorescent response signal.

Upon viral capture, ACE2 enzyme from a liquid reservoir is nebulized into the airstream, and then a buffer rinse is provided to wash away any non-specifically bound ACE2. When virus is present, ACE2 binds to it and is retained. ACE2 is then contacted with a polypeptide substrate, such as 7-methoxycoumarin 4-acetyl-alanine-proline-lysine-2,4-dinitrophenyl hydroxide. The 7-methoxycoumarin acetate (MCA) is a fluorescent molecule, while the 2,4-dinitrophenol (DNP) is a quencher. When covalently attached via a short Ala-Pro-Lys peptide sequence, DNP quenches fluorescence of MCA. If ACE2 is bound to the captured virus, such as SARS-CoV-2 virus, ACE2 will cleave the peptide linkage, spatially separating the DNP quencher and turn on MCA fluorescence. This fluorescence can be read at λex˜322 nm/λem˜381 nm in the near UV.

Similarly, to detect an influenza virus, the present assays exploit its unique viral envelope protein features. Flu virus contains surface hemagglutinin proteins that bind cell surface glycoproteins and facilitate cellular entry. Immobilized Griffithsin captures the flu virus in the airstream via this hemagglutinin. Once captured, the enzymatic activity of another viral surface-bound protein, for example, neuraminidase, can be exploited. Neuraminidase is expressed on the viral envelope to cleave sialylated proteins in mucosal secretions and enable viral replicates to leave the cell. Neuraminidase hydrolyzes a synthetic substrate, MUNANA, producing neuraminic acid, and 4-methylumbelliferone (4-MU), which fluoresces at 440 nm. This chemistry may be employed to continuously monitor air sources for influenza viruses, and also to differentiate coronaviruses such as SARS-CoV-2.

### Diagnostic Viral Assay Kits

Diagnostic kits including one or more of the disclosed Griffithsin peptides and instructions for using the kit in a particular viral detection assay modality also represent preferred aspects of the present disclosure. These kits may further optionally include one or more additional diagnostic compounds, reagents, or any combination thereof.

The kits of the invention may be packaged for commercial distribution and may further optionally include one or more delivery devices adapted to deliver a sample suspected of containing a virus to a sample collection or assay device. Such kits typically include at least one vial, test tube, flask, bottle, syringe, or other container, into which the disclosed composition(s) may be placed, and preferably suitably aliquotted. Where a second distinct diagnostic reagent is also provided, the kit may also contain a second distinct container into which this second distinct diagnostic reagent may be placed. Alternatively, viral detection reagents as described herein may be prepared in a single mixture, such as a suspension or solution, and may be packaged in a single container, such as a vial, flask, syringe, catheter, cannula, bottle, or other suitable single container.

The kits of the present invention may also typically include a retention mechanism adapted to contain or retain the vial(s) or other container(s) in close confinement for commercial sale, such as, e.g., injection or blow-molded plastic containers into which the desired vial(s) or other container(s) may be retained to minimize or prevent breakage, exposure to sunlight, or other undesirable factors, or to permit ready use of the composition(s) included within the kit.

Antibody Compositions

In certain aspects of the invention, it may be desirable to generate anti-viral antibodies or antibodies that specifically bind to one or more unique or novel epitope sequences on a virus of interest. In such applications, the antibodies may be labeled with one or more detectable reagents to facilitate identification and/or quantitation of such compounds in a sample. The present application also concerns methodology for generating candidate viral epitope or anti-viral antibodies and the routine technical aspects of the assays required to identify actual specific antibodies from the pool of candidates. In light of this invention, therefore, a range of suitable antibodies may be made and used in a variety of embodiments, including in the detection of viral particles, viral-peptides, or viral-derived epitopic sequences in a sample.

The use of monoclonal antibodies (MAbs) or derivatives thereof is also preferred in the practice of certain aspects of the present disclosure. MAbs are recognized to have certain advantages, e.g., reproducibility and large-scale production, that makes them suitable for use in diagnostic detection methods. In such embodiments, the invention also provides monoclonal antibodies of the murine, human, monkey, rat, hamster, rabbit and even frog or chicken origin. Murine, human or humanized monoclonal antibodies will generally be preferred.

As will be understood by those in the art, the immunological binding reagents encompassed by the term “antibody” extend to all antibodies from all species, and antigen binding fragments thereof, including dimeric, trimeric and multimeric antibodies; bispecific antibodies; chimeric antibodies; human and humanized antibodies; recombinant and engineered antibodies, and fragments thereof.

Immunology Assays for Detecting Viral Epitopes

While the present disclosure provides certain advantages over conventional detection assays, in certain embodiments, it may be desirable to employ one or more functional assays to identify and/or confirm the identity or presence of one or more viral epitopes, and specifically one or more coronavirus or influenza-specific peptides or polypeptides.

### Methods for Detecting Virus Particles

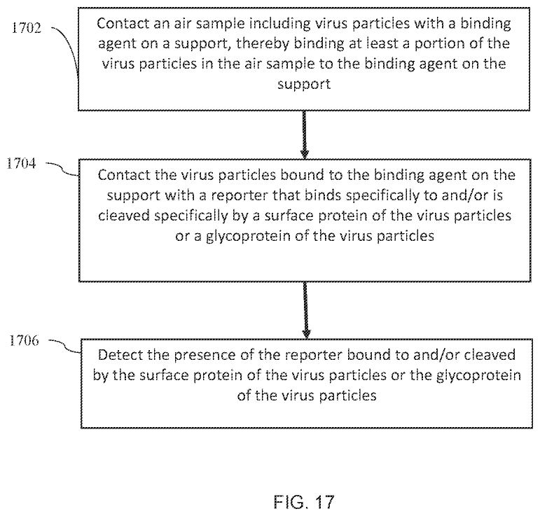

Referring to FIG. 17, a non-limiting embodiment of a method for detecting a virus is provided. At step 1702, the method comprises contacting an air sample including virus particles with a binding agent on a support, thereby binding at least a portion of the virus particles in the air sample to the binding agent on the support. The air sample can be continuously or intermittently provided to the support. For example, an air pump and tubing can be used to direct the air sample to the support. In various non-limiting embodiments, the binding agent can be immobilized on the support prior to contacting the binding agent with the air sample.

The air sample can comprise the virus particles suspended in a gas. For example, the virus particles can be suspended in air. For example, the air sample can comprise at least 95% by weight of air based on the total weight of the air sample. In various embodiments, the virus particles in the air sample can be aerosolized or airborne.

The virus particles can comprise at least one species in at least one genus of Influenzavirus, Coronavirus, or Paramyxovirus. For example, the virus particles can comprise at least one of rubella virus, morbillivirus, pneumovirus, paramyxovirus, a human pathogenic serotype of Influenza A, SARS-CoV-2, and a human pathogenic serotype of Influenza B. In various non-limiting embodiments, the virus particles can comprise at least one of SARS-CoV-2, a mutant thereof, and a derivative thereof.

In various embodiments, the binding agent can be configured to bind to the virus particles such that the virus particles can be immobilized on the support. For example, the binding agent can comprise at least one of a lectin, an antibody, and an antigen-binding fragment. In various non-limiting embodiments, the binding agent comprises a lectin comprising a Griffithsin polypeptide or peptide. For example, the Griffithsin polypeptide or peptide can comprise an amino acid sequence that is at least 98% identical to a sequence of at least fifteen amino acids of SEQ ID NO:1.

The support is configured to maintain the binding agent immobilized on the support while an air sample is contacted with the binding agent and/or support. The support can be impermeable to air or the support can be permeable to air. In various non-limiting embodiments, the support comprises at least one of a microplate and an impaction disk.

Referring again to FIG. 17, at step 1704, the method comprises contacting the virus particles bound to the binding agent on the support with a reporter that binds specifically to and/or is cleaved specifically by a surface protein of the virus particles or a glycoprotein of the virus particles. At step 1706, the method comprises detecting the presence of the reporter bound to and/or cleaved by the surface protein of the virus particles or the glycoprotein of the virus particles.

In various non-limiting embodiments, the surface protein of the virus particles or the glycoprotein of the virus particles comprises at least one of an angiotensin-converting enzyme 2 (ACE-2) protein, a hemagglutinin protein, a spike protein, a neuraminidase polypeptide, and an F protein. For example, the surface protein of the virus particles or the glycoprotein of the virus particles can comprises an ACE-2 protein.

The reporter is configured to enable detection of the virus. For example, the reporter can specifically bind with a surface protein of the virus particles or a glycoprotein of the virus particles. In certain non-limiting embodiments, the reporter can comprise a molecular group that can selectively bind to a surface protein of the virus particles or a glycoprotein of the virus particles, while not binding to certain other types of viruses, biomolecules, and/or chemicals. Therefore, detecting that the reporter is bound to the virus can enable detection of the virus particles. In various non-limiting embodiments, the reporter can comprise a molecular group that can be cleaved selectively by a surface protein of the virus particles or a glycoprotein of the virus particles, while not being cleaved by certain other types of viruses, biomolecules, and/or chemicals. Therefore, detecting the resulting products of the cleaved molecular group can enable detection of the virus particles. In various non-limiting embodiments, binding specifically to and/or cleaved specifically by can comprise binding specifically to and/or being cleaved specifically by the virus particles and at least one other biomolecule (e.g., other virus particles). In certain non-limiting embodiments in which the reporter comprises a substrate for an enzyme and the surface protein of the virus particles comprises an enzyme, detecting the presence of the reporter bound to and/or cleaved by the surface protein of the virus particles or the glycoprotein of the virus particles comprises detecting the presence of a product formed from the substrate in the presence of the enzyme.

In various non-limiting embodiments, the reporter can comprise at least one of a substrate for an enzyme, a lectin bound to an enzyme, a labeled lectin, a labeled antibody, and a labeled antigenic fragment. In various non-limiting embodiments, the reporter comprises a substrate for an enzyme and the substrate comprises at least one of 4-methylumbelli-feryl N-acetyl α-D-neuraminic acid and 7-methoxycoumarin 4-acetyl-alanine-proline-lysine-2,4-dinitrophenyl hydroxide. In certain embodiments, the substrate can be cleaved specifically by the surface protein of the virus particles or the glycoprotein of the virus particles to form 7-methoxycoumarin-4-acetic acid and 2,4-dinitrophenol.

The labeled lectin, the labeled antibody, and/or the labeled antigenic fragment, if present, can comprise at least one of a dye and an enzyme bound to the respective lectin, antibody, and/or antigenic fragment. For example, the labeled lectin, if present, can comprise a dye bound to the lectin, an enzyme bound to the lectin, or both a dye and an enzyme bound to the lectin. The labeled antibody, if present, can comprise a dye bound to the antibody, an enzyme bound to the antibody, or both a dye and an enzyme bound to the antibody. The labeled antigenic fragment, if present, can comprise a dye bound to the antigenic fragment, an enzyme bound to the antigenic fragment, or both a dye and an enzyme bound to the antigenic fragment. The lectin, antibody, and/or antigenic fragment portion, if present, can bind specifically to the surface protein of the virus particles or the glycoprotein of the virus particles, while the dye and/or the enzyme can be detected. For example, the dye can comprise a self-quenched dye, a fluorescent dye, or an electrochemiluminescent dye. In various non-limiting embodiments, detecting the dye can comprise exciting the dye and detecting an emission of the dye. In various non-limiting embodiments, detecting the enzyme can comprise contacting the enzyme with a substrate and detecting a product formed from the substrate in the presence of the enzyme. Detecting the presence of reporter bound to and/or cleaved by the surface protein of the virus particles or the glycoprotein of the virus particles can be performed in no greater than sixty minutes, such as, for example, no greater than forty five minutes, no greater than thirty minutes, no greater than twenty minutes, no greater than ten minutes, no greater than five minutes, or no greater than three minutes. For example, detecting the presence of reporter bound to and/or cleaved by the surface protein of the virus particles or the glycoprotein of the virus particles can be performed in a time period in a range of one second to sixty minutes, such as, for example, ten seconds to forty five minutes, ten second to thirty minutes, thirty seconds to ten minutes, or thirty seconds to five minutes. The method of detecting the virus particles according to the present disclosure can enable a more rapid detection of virus particles than previous methods.

The method according to the present disclosure can have a lower detection limit of no greater than 6,000,000 plaque-forming units (Pfu)/mL, such as, for example, no greater than 100,000 Pfu/ml, no greater than 10,000 Pfu/mL, or no greater than 1,000 Pfu/ml.

### Systems for Detecting Presence of Virus Particles

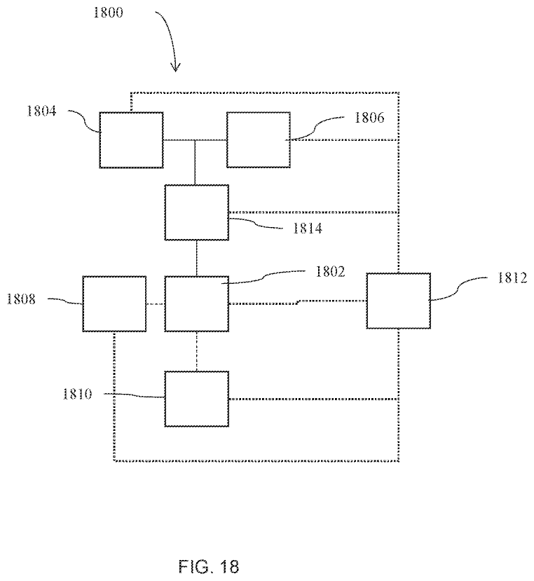

Referring to FIG. 18, a non-limiting embodiment of a system 1800 for detecting the presence of virus particles is provided. The system 1800 comprises a support 1802, an air pump 1804, a fluid delivery apparatus 1806, a sensor, and a programmable hardware device 1812. The support 1802 comprises a binding agent configured to bind at least a portion of virus particles in an air sample. The air pump 1804 can be in fluid communication with the support 1802 and configured to deliver the air sample to the support 1802. For example, the air pump 1804 can be mounted upstream of the support 1802 and direct air towards the support 1802 with a fluid conduit (e.g., tube). In various non-limiting embodiments, the air pump 1804 can be mounted downstream (not shown) of the support 1804 and create a vacuum which draws the air sample through the support 1802.

The fluid delivery apparatus 1806 can be configured to deliver a reporter to the support 1802. For example, the fluid delivery apparatus 1806 can be a nebulizer, and the fluid delivery apparatus 1806 can be in fluid communication with the air pump 1804 and the support 1802 such that the reporter can be aerosolized and/or suspended into an air stream and carried to the support 1802. In various non-limiting embodiments, the air stream can comprise the air sample and optionally an additional volume of a gas. In certain non-limiting embodiments, the fluid delivery apparatus 1806 can comprise a liquid pump in fluid communication with the support 1802. In various non-limiting embodiments, the system 1800 can include at least two fluid delivery apparatus 1806. For example, one fluid delivery apparatus can provide the reporter to the support 1802 and another fluid delivery apparatus can provide a substrate, buffer, and/or other fluid to the support 1802.

The sensor can comprise an electromagnetic radiation source 1808 and an electromagnetic radiation sensor 1810, which can be in optical communication with the support 1802. For example, the electromagnetic radiation source 1808 can be configured to emit electromagnetic radiation directed at the support 1802, and the electromagnetic radiation sensor 1810 can be configured to detect electromagnetic radiation from the support 1802. In certain non-limiting embodiments, the electromagnetic radiation source 1808 can comprise a light emitting diode, a laser diode, and/or other light source. The electromagnetic radiation sensor 1810 can comprise a photodetector. Collectively, the electromagnetic radiation source 1808 and the electromagnetic radiation sensor 1810 can form a spectrophotometer. In various non-limiting embodiments, the sensor can comprise an electrochemical response sensor (e.g., a pH sensor, an oxidation/reduction sensor). For example, an electrochemical response sensor can measure a pH of the support (e.g., liquid on the support).

The programmable hardware device 1812 can be in electrical communication with the electromagnetic radiation source 1808 and the electromagnetic radiation sensor 1810 and configured to measure an electromagnetic property from the support 1802 to thereby determine presence of the reporter on the support 1802. The programmable hardware device 1812 can comprise a processor operatively coupled to memory and/or other electronic hardware as needed to perform measurement of the electromagnetic property.

The programmable hardware device 1812 can be in electrical communication with the air pump 1804 and the fluid delivery apparatus 1806. The system 1800, by the programmable hardware device 1812, can be configured to perform a cycle including cyclically delivering an air sample to the support 1802 with the air pump 1802, delivering the reporter to the support 1802 with the fluid delivery system 1806, measuring the electromagnetic property of the electromagnetic radiation from the support with the electromagnetic radiation source 1808 and the electromagnetic radiation sensor 1810, and other optional steps. In various non-limiting embodiments, the system 1800 can perform the cycle in no longer than five minutes, such as, for example, no longer than three minutes.

In certain non-limiting embodiments, the system 1800 can comprise a light scatter-based particle detector 1814 configured to determine a quantity of particles in the air sample. Based on a threshold quantity of particles in the air sample, the system 1800 can then proceed to detect the presence of virus particles in the air sample using the support 1802. In various non-limiting embodiments, the air sample is from a heating, ventilation and air-conditioning (HVAC) system.

### Exemplary Definitions

In accordance with the present invention, polynucleotides, nucleic acid segments, nucleic acid sequences, and the like, include, but are not limited to, DNAs (including and not limited to genomic or extragenomic DNAs), genes, peptide nucleic acids (PNAs) RNAs (including, but not limited to, rRNAs, mRNAs and tRNAs), nucleosides, and suitable nucleic acid segments either obtained from natural sources, chemically synthesized, modified, or otherwise prepared or synthesized in whole or in part by the hand of man.

Unless defined otherwise, all technical and scientific terms used herein have the same meaning as commonly understood by one of ordinary skill in the art to which this invention belongs. The following references provide one of skill with a general definition of many of the terms used in this invention: Dictionary of Biochemistry and Molecular Biology, (2^{nd} Ed.) J. Stenesh (Ed.), Wiley-Interscience (1989); Dictionary of Microbiology and Molecular Biology (3 rd Ed.), P. Singleton and D. Sainsbury (Eds.), Wiley-Interscience (2007); Chambers Dictionary of Science and Technology (2^{nd} Ed.), P. Walker (Ed.), Chambers (2007); Glossary of Genetics (5^{th} Ed.), R. Rieger et al. (Eds.), Springer-Verlag (1991); and The HarperCollins Dictionary of Biology, W. G. Hale and J. P. Margham, (Eds.), HarperCollins (1991).

Although any methods and compositions similar or equivalent to those described herein can be used in the practice or testing of the present invention, the preferred methods, and compositions are described herein. For purposes of the present invention, the following terms are defined below for sake of clarity and ease of reference:

In accordance with long standing patent law convention, the words “a” and “an,” when used throughout this application and in the claims, denote “one or more.”

The terms “about” and “approximately” as used herein, are interchangeable, and should generally be understood to refer to a range of numbers around a given number, as well as to all numbers in a recited range of numbers (e.g., “about 5 to 15” means “about 5 to about 15” unless otherwise stated). Moreover, all numerical ranges herein should be understood to include each whole integer within the range.

“Biocompatible” refers to a material that, when exposed to living cells, will support an appropriate cellular activity of the cells without causing an undesirable effect in the cells, such as a change in a living cycle of the cells, a change in a proliferation rate of the cells, or a cytotoxic effect.

The term “biologically-functional equivalent” is well understood in the art, and is further defined in detail herein. Accordingly, sequences that have about 85% to about 90%; or more preferably, about 91% to about 95%; or even more preferably, about 96% to about 99%; of nucleotides that are identical or functionally-equivalent to one or more of the nucleotide sequences provided herein are particularly contemplated to be useful in the practice of the methods and compositions set forth in the instant application.

As used herein, “biomimetic” shall mean a resemblance of a synthesized material to a substance that occurs naturally in a human body and which is not rejected by (e.g., does not cause an adverse reaction in) the human body.

As used herein, the term “buffer” includes one or more compositions, or aqueous solutions thereof, that resist fluctuation in the pH when an acid or an alkali is added to the solution or composition that includes the buffer. This resistance to pH change is due to the buffering properties of such solutions, and may be a function of one or more specific compounds included in the composition. Thus, solutions or other compositions exhibiting buffering activity are referred to as buffers or buffer solutions. Buffers generally do not have an unlimited ability to maintain the pH of a solution or composition; rather, they are typically able to maintain the pH within certain ranges, for example from a pH of about 5 to 7.

As used herein, the term “carrier” is intended to include any solvent(s), dispersion medium, coating(s), diluent(s), buffer(s), isotonic agent(s), solution(s), suspension(s), colloid(s), inert(s) or such like, or a combination thereof, that is pharmaceutically acceptable for administration to the relevant animal. The use of one or more delivery vehicles for chemical compounds in general, and chemotherapeutics in particular, is well known to those of ordinary skill in the pharmaceutical arts. Except insofar as any conventional media or agent is incompatible with the active ingredient, its use in the diagnostic, prophylactic, and therapeutic compositions is contemplated. One or more supplementary active ingredient(s) may also be incorporated into, or administered in association with, one or more of the disclosed chemotherapeutic compositions.

As used herein, “COVID-19,” “SARS-CoV-2,” and “novel 2019 coronavirus” are meant to be interchangeable.

As used herein, the term “DNA segment” refers to a DNA molecule that has been isolated free of total genomic DNA of a particular species. Therefore, a DNA segment obtained from a biological sample using one of the compositions disclosed herein refers to one or more DNA segments that have been isolated away from, or purified free from, total genomic DNA of the particular species from which they are obtained. Included within the term “DNA segment,” are DNA segments and smaller fragments of such segments, as well as recombinant vectors, including, for example, plasmids, cosmids, phage, viruses, and the like.

The term “effective amount,” as used herein, refers to an amount that is capable of treating or ameliorating a disease or condition or otherwise capable of producing an intended therapeutic effect.

The term “for example” or “e.g.,” as used herein, is used merely by way of example, without limitation intended, and should not be construed as referring only those items explicitly enumerated in the specification.

As used herein, a “heterologous” sequence is defined in relation to a predetermined, reference sequence, such as, a polynucleotide or a polypeptide sequence. For example, with respect to a structural gene sequence, a heterologous promoter is defined as a promoter which does not naturally occur adjacent to the referenced structural gene, but which is positioned by laboratory manipulation. Likewise, a heterologous gene or nucleic acid segment is defined as a gene or segment that does not naturally occur adjacent to the referenced promoter and/or enhancer elements.

As used herein, “homologous” means, when referring to polynucleotides, sequences that have the same essential nucleotide sequence, despite arising from different origins. Typically, homologous nucleic acid sequences are derived from closely related genes or organisms possessing one or more substantially similar genomic sequences. By contrast, an “analogous” polynucleotide is one that shares the same function with a polynucleotide from a different species or organism but may have a significantly different primary nucleotide sequence that encodes one or more proteins or polypeptides that accomplish similar functions or possess similar biological activity. Analogous polynucleotides may often be derived from two or more organisms that are not closely related (e.g., either genetically or phylogenetically).

As used herein, the term “homology” refers to a degree of complementarity between two or more polynucleotide or polypeptide sequences. The word “identity” may substitute for the word “homology” when a first nucleic acid or amino acid sequence has the exact same primary sequence as a second nucleic acid or amino acid sequence. Sequence homology and sequence identity can be determined by analyzing two or more sequences using algorithms and computer programs known in the art. Such methods may be used to assess whether a given sequence is identical or homologous to another selected sequence.

As used herein, “host cell receptor,” “host receptor protein,” “viral host receptor specific polypeptide” and “ligand” are meant to be interchangeable. The terms “identical” or percent “identity,” in the context of two or more nucleic acid or polypeptide sequences, refer to two or more sequences or subsequences that are the same or have a specified percentage of amino acid residues or nucleotides that are the same, when compared and aligned for maximum correspondence, as measured using one of the sequence comparison algorithms described below (or other algorithms available to persons of ordinary skill) or by visual inspection.

As used herein, the phrase “in need of treatment” refers to a judgment made by a caregiver such as a physician or veterinarian that a patient requires (or will benefit in one or more ways) from treatment. Such judgment may made based on a variety of factors that are in the realm of a caregiver's expertise and may include the knowledge that the patient is ill as the result of a disease state that is treatable by one or more compound or pharmaceutical compositions such as those set forth herein.

The phrases “isolated” or “biologically pure” refer to material that is substantially, or essentially, free from components that normally accompany the material as it is found in its native state.

As used herein, the term “kit” may be used to describe variations of a portable, self-contained enclosure or commercially-packaged article of manufacture that includes at least one set of reagents, components, or diagnostically-formulated compositions to conduct one or more of the viral detection and assay methods of the present disclosure. Optionally, such kit may include one or more sets of instructions for use of the enclosed reagents, such as, for example, in a laboratory in the field, at a distant or remote location, on-site, in a hospital or a clinical laboratory or such like.

“Link” or “join” refers to any method known in the art for functionally connecting one or more proteins, peptides, nucleic acids, or polynucleotides, including, without limitation, recombinant fusion, covalent bonding, disulfide bonding, ionic bonding, hydrogen bonding, electrostatic bonding, and the like.

The term “naturally-occurring” as used herein as applied to an object refers to the fact that an object can be found in nature. For example, a polypeptide or polynucleotide sequence that is present in an organism (including one or more influenzaviruses, coronaviruses, and the like) that can be isolated from a source in nature and which has not been intentionally modified by the hand of man in a laboratory is naturally-occurring.

As used herein, the term “nucleic acid” includes one or more types of: polydeoxyribonucleotides (containing 2-deoxy-D-ribose), polyribonucleotides (containing D-ribose), and any other type of polynucleotide that is an N-glycoside of a purine or pyrimidine base, or modified purine or pyrimidine bases (including abasic sites). The term “nucleic acid,” as used herein, also includes polymers of ribonucleosides or deoxyribonucleosides that are covalently bonded, typically by phosphodiester linkages between subunits, but in some cases by phosphorothioates, methylphosphonates, and the like. “Nucleic acids” include single- and double-stranded DNA, as well as single- and double-stranded RNA. Exemplary nucleic acids include, without limitation, gDNA; hnRNA; mRNA; rRNA, tRNA, micro RNA (miRNA), small interfering RNA (siRNA), small nucleolar RNA (snORNA), small nuclear RNA (snRNA), and small temporal RNA (stRNA), and the like, and any combination thereof.

The terms “operably linked” and operatively linked,” as used herein, refers to that union of the nucleic acid sequences that are linked in such a way, such that the coding regions are contiguous and in correct reading frame. Such sequences are typically contiguous, or substantially contiguous. However, since enhancers generally function when separated from the promoter by several kilobases and intronic sequences may be of variable lengths, some polynucleotide elements may be operably linked but not contiguous.

As used herein, the term “patient” (also interchangeably referred to as “host” or “subject”) refers to any host that can receive one or more of the pharmaceutical compositions disclosed herein. Preferably, the subject is a vertebrate animal, which is intended to denote any animal species (and preferably, a mammalian species such as a human being). In certain embodiments, a “patient” refers to any animal host including without limitation any mammalian host. Preferably, the term refers to any mammalian host, the latter including but not limited to, human and non-human primates, bovines, canines, caprines, cavines, corvines, epines, equines, felines, hircines, lapines, leporines, lupines, murines, ovines, porcines, ranines, racines, vulpines, and the like, including livestock, zoological specimens, exotics, as well as companion animals, pets, and any animal under the care of a veterinary practitioner. A patient can be of any age at which the patient is able to respond to inoculation with the present vaccine by generating an immune response. In particular embodiments, the mammalian subject is preferably human.

The phrase “pharmaceutically-acceptable” refers to molecular entities and compositions that preferably do not produce an allergic or similar untoward reaction when administered to a mammal, and in particular, when administered to a human.

As used herein, “pharmaceutically acceptable salt” refers to a salt that preferably retains the desired biological activity of the parent compound and does not impart any undesired toxicological effects. Examples of such salts include, without limitation, acid addition salts formed with inorganic acids (e.g., hydrochloric acid, hydrobromic acid, sulfuric acid, phosphoric acid, nitric acid, and the like); and salts formed with organic acids including, without limitation, acetic acid, oxalic acid, tartaric acid, succinic acid, maleic acid, fumaric acid, gluconic acid, citric acid, malic acid, ascorbic acid, benzoic acid, tannic acid, pamoic (embonic) acid, alginic acid, naphthoic acid, polyglutamic acid, naphthalenesulfonic acids, naphthalenedisulfonic acids, polygalacturonic acid; salts with polyvalent metal cations such as zinc, calcium, bismuth, barium, magnesium, aluminum, copper, cobalt, nickel, cadmium, and the like; salts formed with an organic cation formed from N,N′-dibenzylethylenediamine or ethylenediamine; and combinations thereof.

As used herein, the term “plasmid” or “vector” refers to a genetic construct that is composed of genetic material (i.e., nucleic acids). Typically, a plasmid or a vector contains an origin of replication that is functional in bacterial host cells, e.g., Escherichia coli, and selectable markers for detecting bacterial host cells including the plasmid.

Plasmids and vectors of the present invention may include one or more genetic elements as described herein arranged such that an inserted coding sequence can be transcribed and translated in a suitable expression cells. In addition, the plasmid or vector may include one or more nucleic acid segments, genes, promoters, enhancers, activators, multiple cloning regions, or any combination thereof, including segments that are obtained from or derived from one or more natural and/or artificial sources.

As used herein, “polymer” means a chemical compound or mixture of compounds formed by polymerization and including repeating structural units. Polymers may be constructed in multiple forms and compositions or combinations of compositions.

As used herein, the term “polypeptide” is intended to encompass a singular “polypeptide” as well as plural “polypeptides,” and includes any chain or chains of two or more amino acids. Thus, as used herein, terms including, but not limited to “peptide,” “dipeptide,” “tripeptide,” “protein,” “enzyme,” “amino acid chain,” and “contiguous amino acid sequence” are all encompassed within the definition of a “polypeptide,” and the term “polypeptide” can be used instead of, or interchangeably with, any of these terms. The term further includes polypeptides that have undergone one or more post-translational modification(s), including for example, but not limited to, glycosylation, acetylation, phosphorylation, amidation, derivatization, proteolytic cleavage, post-translation processing, or modification by inclusion of one or more non-naturally occurring amino acids. Conventional nomenclature exists in the art for polynucleotide and polypeptide structures.

For example, one-letter and three-letter abbreviations are widely employed to describe amino acids: Alanine (A; Ala), Arginine (R; Arg), Asparagine (N; Asn), Aspartic Acid (D; Asp), Cysteine (C; Cys), Glutamine (Q; Gln), Glutamic Acid (E; Glu), Glycine (G; Gly), Histidine (H; His), Isoleucine (I; Ile), Leucine (L; Leu), Methionine (M; Met), Phenylalanine (F; Phe), Proline (P; Pro), Serine (S; Ser), Threonine (T; Thr), Tryptophan (W; Trp), Tyrosine (Y; Tyr), Valine (V; Val), and Lysine (K; Lys). Amino acid residues described herein are preferred to be in the “L” isomeric form. However, residues in the “D” isomeric form may be substituted for any L-amino acid residue provided the desired properties of the polypeptide are retained.

As used herein, the terms “prevent,” “preventing,” “prevention,” “suppress,” “suppressing,” and “suppression” as used herein refer to administering a compound either alone or as contained in a pharmaceutical composition prior to the onset of clinical symptoms of a disease state so as to prevent any symptom, aspect or characteristic of the disease state. Such preventing and suppressing need not be absolute to be deemed medically useful.

“Protein” is used herein interchangeably with “peptide” and “polypeptide,” and includes both peptides and polypeptides produced synthetically, recombinantly, or in vitro and peptides and polypeptides expressed in vivo after nucleic acid sequences are administered into a host animal or human subject. The term “polypeptide” is preferably intended to refer to any amino acid chain length, including those of short peptides from about 2 to about 20 amino acid residues in length, oligopeptides from about 10 to about 100 amino acid residues in length, and longer polypeptides including from about 100 amino acid residues or more in length. Furthermore, the term is also intended to include enzymes, i.e., functional biomolecules including at least one amino acid polymer. Polypeptides and proteins of the present invention also include polypeptides and proteins that are or have been post-translationally modified and include any sugar or other derivative(s) or conjugate(s) added to the backbone amino acid chain.

“Purified,” as used herein, means separated from many other compounds or entities. A compound or entity may be partially purified, substantially purified, or pure. A compound or entity is considered pure when it is removed from substantially all other compounds or entities, i.e., is preferably at least about 90%, more preferably at least about 91%, 92%, 93%, 94%, 95%, 96%, 97%, 98%, 99%, or greater than 99% pure. A partially or substantially purified compound or entity may be removed from at least 50%, at least 60%, at least 70%, or at least 80% of the material with which it is naturally found, e.g., cellular material such as cellular proteins and/or nucleic acids.

The term “recombinant” indicates that the material (e.g., a polynucleotide or a polypeptide) has been artificially or synthetically (non-naturally) altered by human intervention. The alteration can be performed on the material within or removed from, its natural environment, or native state. Specifically, e.g., a promoter sequence is “recombinant” when it is produced by the expression of a nucleic acid segment engineered by the hand of man. For example, a “recombinant nucleic acid” is one that is made by recombining nucleic acids, e.g., during cloning, DNA shuffling or other procedures, or by chemical or other mutagenesis; a “recombinant polypeptide” or “recombinant protein” is a polypeptide or protein which is produced by expression of a recombinant nucleic acid; and a “recombinant virus,” e.g., a recombinant AAV virus, is produced by the expression of a recombinant nucleic acid.

The term “regulatory element,” as used herein, refers to a region or regions of a nucleic acid sequence that regulates transcription. Exemplary regulatory elements include, but are not limited to, enhancers, post-transcriptional elements, transcriptional control sequences, and such like.

The term “RNA segment” refers to an RNA molecule that has been isolated free of total cellular RNA of a particular species. Therefore, RNA segments can refer to one or more RNA segments (either of native or synthetic origin) that have been isolated away from, or purified free from, other RNAs. Included within the term “RNA segment,” are RNA segments and smaller fragments of such segments.

As used herein, “SARS,” “SARS-CoV,” and “SARS coronavirus XXX” are meant to be interchangeable.

The term “a sequence essentially as set forth in SEQ ID NO:X” means that the sequence substantially corresponds to a portion of SEQ ID NO:X and has relatively few nucleotides (or amino acids in the case of polypeptide sequences) that are not identical to, or a biologically functional equivalent of, the nucleotides (or amino acids) of SEQ ID NO:X. The term “biologically functional equivalent” is well understood in the art, and is further defined in detail herein. Accordingly, sequences that have about 85% to about 90%; or more preferably, about 91% to about 95%; or even more preferably, about 96% to about 99%; of nucleotides that are identical or functionally equivalent to one or more of the nucleotide sequences provided herein are particularly contemplated to be useful in the practice of the invention.

Suitable standard hybridization conditions for nucleic acids for use in the present invention include, for example, hybridization in 50% formamide, 5×Denhardt's solution, 5×SSC, 25 mM sodium phosphate, 0.1% SDS and 100 μg/mL of denatured salmon sperm DNA at 42° C. for 16 hr followed by 1 hr sequential washes with 0.1×SSC, 0.1% SDS solution at 60° C. to remove the desired amount of background signal. Lower stringency hybridization conditions for the present invention include, for example, hybridization in 35% formamide, 5×Denhardt's solution, 5×SSC, 25 mM sodium phosphate, 0.1% SDS and 100 μg/mL denatured salmon sperm DNA or E. coli DNA at 42° C. for 16 hr followed by sequential washes with 0.8×SSC, 0.1% SDS at Those of ordinary skill in the art will recognize that such hybridization conditions can be readily adjusted to obtain the desired level of stringency for a particular application.

As used herein, the term “structural gene” is intended to generally describe a polynucleotide, such as a gene, that is expressed to produce an encoded peptide, polypeptide, protein, ribozyme, catalytic RNA molecule, or antisense molecule.

The term “subject,” as used herein, describes an organism, including mammals such as primates, to which treatment with the compositions according to the present invention can be provided. Mammalian species that can benefit from the disclosed methods of treatment include, but are not limited to, apes; chimpanzees; orangutans; humans; monkeys; domesticated animals such as dogs and cats; livestock such as horses, cattle, pigs, sheep, goats, and chickens; and other animals such as mice, rats, guinea pigs, and hamsters.

The term “substantially complementary,” when used to define either amino acid or nucleic acid sequences, means that a particular subject sequence, for example, an oligonucleotide sequence, is substantially complementary to all or a portion of the selected sequence, and thus will specifically bind to a portion of an mRNA encoding the selected sequence. As such, typically the sequences will be highly complementary to the mRNA “target” sequence, and will have no more than about 1, about 2, about 3, about 4, about 5, about 6, about 7, about 8, about 9, or about 10 or so base mismatches throughout the complementary portion of the sequence. In many instances, it may be desirable for the sequences to be exact matches, i.e., be completely complementary to the sequence to which the oligonucleotide specifically binds, and therefore have zero mismatches along the complementary stretch. As such, highly complementary sequences will typically bind quite specifically to the target sequence region of the mRNA and will therefore be highly efficient in reducing, and/or even inhibiting the translation of the target mRNA sequence into polypeptide product.

Substantially complementary nucleic acid sequences will be greater than about 80 percent complementary (or “% exact-match”) to a corresponding nucleic acid target sequence to which the nucleic acid specifically binds, and will, more preferably be greater than about 85 percent complementary to the corresponding target sequence to which the nucleic acid specifically binds. In certain aspects, as described above, it will be desirable to have even more substantially complementary nucleic acid sequences for use in the practice of the invention, and in such instances, the nucleic acid sequences will be greater than about 90 percent complementary to the corresponding target sequence to which the nucleic acid specifically binds, and may in certain embodiments be greater than about 95 percent complementary to the corresponding target sequence to which the nucleic acid specifically binds, and even up to and including about 96%, about 97%, about 98%, about 99%, and even about 100% exact match complementary to all or a portion of the target sequence to which the designed nucleic acid specifically binds.

Percent similarity or percent complementary of any of the disclosed nucleic acid sequences may be determined, for example, by comparing sequence information using the GAP computer program, version 6.0, available from the University of Wisconsin Genetics Computer Group (UWGCG). The GAP program utilizes the alignment method of Needleman and Wunsch (1970). Briefly, the GAP program defines similarity as the number of aligned symbols (i.e., nucleotides or amino acids) that are similar, divided by the total number of symbols in the shorter of the two sequences. The preferred default parameters for the GAP program include: (1) a unary comparison matrix (containing a value of 1 for identities and 0 for non-identities) for nucleotides, and the weighted comparison matrix of Gribskov and Burgess (1986), (2) a penalty of 3.0 for each gap and an additional 0.10 penalty for each symbol in each gap; and (3) no penalty for end gaps.

As used herein, the term “substantially free” or “essentially free” in connection with the amount of a component preferably refers to a composition that contains less than about 10 weight percent, preferably less than about 5 weight percent, and more preferably less than about 1 weight percent of a compound. In preferred embodiments, these terms refer to less than about 0.5 weight percent, less than about 0.1 weight percent, or less than about 0.01 weight percent.

As used herein, the term “structural gene” is intended to generally describe a polynucleotide, such as a gene, that is expressed to produce an encoded peptide, polypeptide, protein, ribozyme, catalytic RNA molecule, or antisense molecule.

The term “subject,” as used herein, describes an organism, including mammals such as primates, to which treatment with the compositions according to the present invention can be provided. Mammalian species that can benefit from the disclosed methods of treatment include, but are not limited to, humans, non-human primates such as apes; chimpanzees; monkeys, and orangutans, domesticated animals, including dogs and cats, as well as livestock such as horses, cattle, pigs, sheep, and goats, or other mammalian species including, without limitation, mice, rats, guinea pigs, rabbits, hamsters, and the like.

The terms “substantially corresponds to,” “substantially homologous,” or “substantial identity,” as used herein, denote characteristics of a nucleic acid or an amino acid sequence, wherein a selected nucleic acid sequence or a selected amino acid sequence has at least about 70 or about 75 percent sequence identity as compared to a selected reference nucleic acid or amino acid sequence. More typically, the selected sequence and the reference sequence will have at least about 76, 77, 78, 79, 80, 81, 82, 83, 84 or even 85 percent sequence identity, and more preferably, at least about 86, 87, 88, 89, 90, 91, 92, 93, 94, or 95 percent sequence identity. More preferably still, highly homologous sequences often share greater than at least about 96, 97, 98, or 99 percent sequence identity between the selected sequence and the reference sequence to which it was compared.

As used herein, “synthetic” shall mean that the material is not of a human or animal origin.

As understood herein, a “targeting moiety” is any factor that may facilitate targeting of a specific site by a particle. For example, the targeting moiety may be a chemical targeting moiety, a physical targeting moiety, a geometrical targeting moiety, or a combination thereof. The chemical targeting moiety may be a chemical group or molecule on a surface of the particle; the physical targeting moiety may be a specific physical property of the particle, such as a surface such or hydrophobicity; the geometrical targeting moiety includes a size and a shape of the particle. Further, the chemical targeting moiety may be a dendrimer, an antibody, an aptamer, which may be a thioaptamer, a ligand, an antibody, or a biomolecule that binds a particular receptor on the targeted site. A physical targeting moiety may be a surface charge. The charge may be introduced during the fabrication of the particle by using a chemical treatment such as a specific wash.

For example, immersion of porous silica or oxidized silicon surface into water may lead to an acquisition of a negative charge on the surface. The surface charge may be also provided by an additional layer or by chemical chains, such as polymer chains, on the surface of the particle. For example, polyethylene glycol chains may be a source of a negative charge on the surface. Polyethylene glycol chains may be coated or covalently coupled to the surface using methods known to those of ordinary skill in the art.

The term “diagnostically-practical period” means a period of time that is necessary for one or more of the viral detection agents disclosed herein to be diagnostically effective in identifying the presence of one or more viral isolates in a sample, including, for example, an air sample. Similarly, the term “therapeutically-effective period” refers to a period of time that is necessary for one or more active agents to exert a therapeutic benefit, or to be effective in reducing in severity and/or frequency at least one or more symptoms, or to eliminate one or more symptoms and/or underlying causes, or to be effective in the prevention of an occurrence of one or more symptoms of a disease or infection, and/or their underlying cause, and the improvement or a remediation of damage.

As used herein, a “transcription factor recognition site” and a “transcription factor binding site” refer to a polynucleotide sequence(s) or sequence motif(s), which are identified as being sites for the sequence-specific interaction of one or more transcription factors, frequently taking the form of direct protein-DNA binding. Typically, transcription factor binding sites can be identified by DNA footprinting, gel mobility shift assays, and the like, and/or can be predicted based on known consensus sequence motifs, or by other methods known to those of ordinary skill in the art.

“Transcriptional regulatory element” refers to a polynucleotide sequence that activates transcription alone or in combination with one or more other nucleic acid sequences. A transcriptional regulatory element can, for example, comprise one or more promoters, one or more response elements, one or more negative regulatory elements, and/or one or more enhancers.

“Transcriptional unit” refers to a polynucleotide sequence that comprises at least a first structural gene operably linked to at least a first cis-acting promoter sequence and optionally linked operably to one or more other cis-acting nucleic acid sequences necessary for efficient transcription of the structural gene sequences, and at least a first distal regulatory element as may be required for the appropriate tissue-specific and developmental transcription of the structural gene sequence operably positioned under the control of the promoter and/or enhancer elements, as well as any additional cis-sequences that are necessary for efficient transcription and translation (e.g., polyadenylation site(s), mRNA stability controlling sequence(s), etc.

As used herein, the term “transformation” is intended to generally describe a process of introducing an exogenous polynucleotide sequence (e.g., a viral vector, a plasmid, or a recombinant DNA or RNA molecule) into a host cell or protoplast in which the exogenous polynucleotide is incorporated into at least a first chromosome or is capable of autonomous replication within the transformed host cell. Transfection, electroporation, and “naked” nucleic acid uptake all represent examples of techniques used to transform a host cell with one or more polynucleotides.

As used herein, the term “transformed cell” is intended to mean a host cell whose nucleic acid complement has been altered by the introduction of one or more exogenous polynucleotides into that cell.

“Treating” or “treatment of” as used herein, refers to providing any type of medical or surgical management to a subject. Treating can include, but is not limited to, administering a composition comprising a therapeutic agent to a subject. “Treating” includes any administration or application of a compound or composition of the invention to a subject for purposes such as curing, reversing, alleviating, reducing the severity of, inhibiting the progression of, or reducing the likelihood of a disease, disorder, or condition or one or more symptoms or manifestations of a disease, disorder, or condition. In certain aspects, the compositions of the present invention may also be administered prophylactically, i.e., before development of any symptom or manifestation of the condition, where such prophylaxis is warranted. Typically, in such cases, the subject will be one that has been diagnosed for being “at risk” of developing such a disease or disorder, either as a result of familial history, medical record, or the completion of one or more diagnostic or prognostic tests indicative of a propensity for subsequently developing such a disease or disorder.

As used herein, “a user” is defined as an individual that wishes to determine whether he/she, or some other individual, is infected with a virus, such as an influenza virus, a SARS-CoV, a SARS-CoV-2 virus, or a viral species that is genetically- and/or phylogenetically-related to one or more such viruses. Thus, a user includes, without limitation, front-line workers such as Emergency Medical Technicians (EMTs), police officers, firemen, healthcare workers, medical professional, doctors, nurses, medical technicians, or any other individual wishing to determine viral status for themselves or others.

The term “vector,” as used herein, refers to a nucleic acid molecule (typically comprised of DNA) capable of replication in a host cell and/or to which another nucleic acid segment can be operatively linked so as to bring about replication of the attached segment. A plasmid, cosmid, or a virus is an exemplary vector.

In certain embodiments, it will be advantageous to employ one or more nucleic acid segments of the present invention in combination with an appropriate detectable marker (i.e., a “label,”), such as in the case of employing labeled polynucleotide probes in determining the presence of a given target sequence in a hybridization assay. A wide variety of appropriate indicator compounds and compositions are known in the art for labeling oligonucleotide probes, including, without limitation, fluorescent, radioactive, enzymatic or other ligands, such as avidin/biotin, etc., which are capable of being detected in a suitable assay. In particular embodiments, one may also employ one or more fluorescent labels or an enzyme tag such as urease, alkaline phosphatase or peroxidase, instead of radioactive or other environmentally less-desirable reagents. In the case of enzyme tags, colorimetric, chromogenic, or fluorogenic indicator substrates are known that can be employed to provide a method for detecting the sample that is visible to the human eye, or by analytical methods such as scintigraphy, fluorimetry, spectrophotometry, and the like, to identify specific hybridization with samples containing one or more complementary or substantially complementary nucleic acid sequences. In the case of so-called “multiplexing” assays, where two or more labeled probes are detected either simultaneously or sequentially, it may be desirable to label a first oligonucleotide probe with a first label having a first detection property or parameter (for example, an emission and/or excitation spectral maximum), which also labeled a second oligonucleotide probe with a second label having a second detection property or parameter that is different (i.e., discreet or discernible from the first label. The use of multiplexing assays, particularly in the context of genetic amplification/detection protocols are well-known to those of ordinary skill in the molecular genetic arts.

Biological Functional Equivalents

Modification and changes may be made in the structure of the nucleic acids, or to the vectors comprising them, as well as to mRNAs, polypeptides, or therapeutic agents encoded by them and still obtain functional systems that contain one or more diagnostic, prophylactic, and/or therapeutic agents with desirable characteristics. As mentioned above, it is often desirable to introduce one or more mutations into a specific polynucleotide sequence. In certain circumstances, the resulting encoded polypeptide sequence is altered by this mutation, or in other cases, the sequence of the polypeptide is unchanged by one or more mutations in the encoding polynucleotide.

When it is desirable to alter the amino acid sequence of a polypeptide to create an equivalent, or even an improved, second-generation molecule, the amino acid changes may be achieved by changing one or more of the codons of the encoding DNA sequence, according to Table 1.

For example, certain amino acids may be substituted for other amino acids in a protein structure without appreciable loss of interactive binding capacity with structures such as, for example, antigen-binding regions of antibodies or binding sites on substrate molecules. Since it is the interactive capacity and nature of a protein that defines that protein's biological functional activity, certain amino acid sequence substitutions can be made in a protein sequence, and, of course, its underlying DNA coding sequence, and nevertheless obtain a protein with like properties. It is thus contemplated by the inventors that various changes may be made in the peptide sequences of the disclosed compositions or corresponding DNA sequences which encode said peptides without appreciable loss of their biological utility or activity.

TABLE 1

| AMINO ACIDS | | | CODONS | | | | | |
|---|---|---|---|---|---|---|---|---|
| Alanine | Ala | A | GCA | GCC | GCG | GCU | | |
| Cysteine | Cys | C | UGC | UGU | | | | |
| Aspartic acid | Asp | D | GAC | GAU | | | | |
| Glutamic acid | Glu | E | GAA | GAG | | | | |
| Phenylalanine | Phe | F | UUC | UUU | | | | |
| Glycine | Gly | G | GGA | GGC | GGG | GGU | | |
| Histidine | His | H | CAC | CAU | | | | |
| Isoleucine | Ile | I | AUA | AUC | AUU | | | |
| Lysine | Lys | K | AAA | AAG | | | | |
| Leucine | Leu | L | UUA | UUG | CUA | CUC | CUG | CUU |
| Methionine | Met | M | AUG | | | | | |
| Asparagine | Asn | N | AAC | AAU | | | | |
| Proline | Pro | P | CCA | CCC | CCG | CCU | | |
| Glutamine | Gln | Q | CAA | CAG | | | | |
| Arginine | Arg | R | AGA | AGG | CGA | CGC | CGG | CGU |
| Serine | Ser | S | AGC | AGU | UCA | UCC | UCG | UCU |
| Threonine | Thr | T | ACA | ACC | ACG | ACU | | |
| Valine | Val | V | GUA | GUC | GUG | GUU | | |
| Tryptophan | Trp | W | UGG | | | | | |
| Tyrosine | Tyr | Y | UAC | UAU | | | | |

In making such changes, the hydropathic index of amino acids may be considered. The importance of the hydropathic amino acid index in conferring interactive biologic function on a protein is generally understood in the art (Kyte and Doolittle, 1982, incorporate herein by reference). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like. Each amino acid has been assigned a hydropathic index based on its hydrophobicity and charge characteristics (Kyte and Doolittle, 1982). These values are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is known in the art that certain amino acids may be substituted by other amino acids having a similar hydropathic index or score and still result in a protein with similar biological activity, i.e. still obtain a biological functionally equivalent protein. In making such changes, the substitution of amino acids whose hydropathic indices are within ±2 is preferred, those within ±1 are particularly preferred, and those within ±0.5 are even more particularly preferred. It is also understood in the art that the substitution of like amino acids can be made effectively based on hydrophilicity. U.S. Pat. No. 4,554,101 (specifically incorporated herein in its entirety by express reference thereto), states that the greatest local average hydrophilicity of a protein, as governed by the hydrophilicity of its adjacent amino acids, correlates with a biological property of the protein.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent protein. In such changes, the substitution of amino acids whose hydrophilicity values are within ±2 is preferred, those within ±1 are particularly preferred, and those within ±0.5 are even more particularly preferred.

As outlined above, amino acid substitutions are generally therefore based on the relative similarity of the amino acid side-chain substituents, for example, their hydrophobicity, hydrophilicity, charge, size, and the like. Exemplary substitutions that take one or more of the foregoing characteristics into consideration are well known to those of ordinary skill in the art, and include arginine and lysine; glutamate and aspartate; serine and threonine; glutamine and asparagine; and valine, leucine and isoleucine.

The section headings used throughout are for organizational purposes only and are not to be construed as limiting the subject matter described. All documents, or portions of documents, cited in this application (including, but not limited to, patents, patent applications, articles, books, and treatises) are expressly incorporated herein in their entirety by express reference thereto. In the event that one or more of the incorporated literature and similar materials defines a term in a manner that contradicts the definition of that term in this application, this application controls.

### EXAMPLES

The following examples are included to demonstrate illustrative embodiments of the invention. It should be appreciated by those of ordinary skill in the art that the techniques disclosed in these examples represent techniques discovered to function well in the practice of the invention, and thus can be considered to constitute preferred modes for its practice. However, those of ordinary skill in the art should, in light of the present disclosure appreciate that many changes can be made in the specific embodiments which are disclosed and still obtain a like or similar result without departing from the spirit and scope of the invention.

### Example 1—Methods for Capturing Air Samples for Viral Pathogen Screening

This example demonstrates highly-specific fluorescent kinetic enzyme assays that sensitively detect and differentiate the SARS-CoV-2 and influenza (flu) viruses for secondary screening and diagnosis of infection in public venues. Leveraging prior implementation of enzymatic chemistry demonstrated to detect nerve agent vapors and aerosols with unprecedented sensitivity facilitated the development of rapid fluorescent assays for both the SARS-CoV-2 virus and the flu virus for early detection of viral pathogens in the air. Built-in amplification from targeting surface enzyme chemistries on viral pathogens will provide a game-changing capability to detect viral aerosols in real time, which would enable detection of disease outbreaks before achieving epidemic proportions. These chemistries can be implemented in several form factors (air monitors for crowded public spaces or individual, exhaled-air test kits) that can be deployed for rapid public area screening or individual testing.

Presently, the lack of ability to detect viruses in real time presents a substantial challenge to the US homeland and limits our ability to rapidly identify people infected with potentially virulent pathogens and to limit the spread of infection to prevent outbreaks as quickly as possible. The current state-of-the-art in the detection of COVID-19 infection relies upon nasopharyngeal swab sampling coupled with desktop clinical chemistry tests, which typically rely upon reverse transcription-polymerase chain reaction (RT-PCR) to detect viral RNA. Presently, numerous companies are obtaining FDA approval for PCR-based tests that provide results in five minutes. However, this testing paradigm does not address some of the significant challenges associated with detecting COVID-19 rapidly in large populations of people. Many carriers of SARS-CoV-2 can be asymptomatic, but still transmit the virus to others. This problem makes detection of COVID-19 in public places nearly impossible. Diagnosis of infection, contact tracing, and quarantining are presently the standard for limiting the spread of infection; however, the delays between transmission and symptom onset enable the spread of the disease in today's global society.

Presently, many public places such as airports and retail stores, are seeking to implement the use of infrared (IR) cameras for elevated skin temperature (EST) screening. IR cameras image the facial temperature of people queued through a security checkpoint or entry. Those with temperatures above 100.4° F. are flagged as potentially having a fever and escorted to a secondary health screening site where a diagnostic test is administered to diagnose a COVID-19 infection. This technique will miss people who are asymptomatic and able to transmit the disease, presenting a critical gap in the lag between spread and detection that can make the difference, as the current pandemic has proven. Further, invasive swab sampling also exposes medical personnel to risk and demands a significant burden for use of personalized protective equipment (PPE).

A superior approach to rapid viral detection for real-time prevention of disease spread would leverage chemistry that correctly detects unique surface markers of the virus and amplifies their presence in an easy-to-read colorimetric or fluorescent test. Such chemistry could be deployed in several form factors, ranging from an active air sampling device that automatically assays for viruses, or as a small handheld device that can sample an individual's exhaled breath (non-contact) and develop a quick signal analysis enabling confirmation of test results in less than five minutes while requiring no sample processing or exposure via contact sampling.

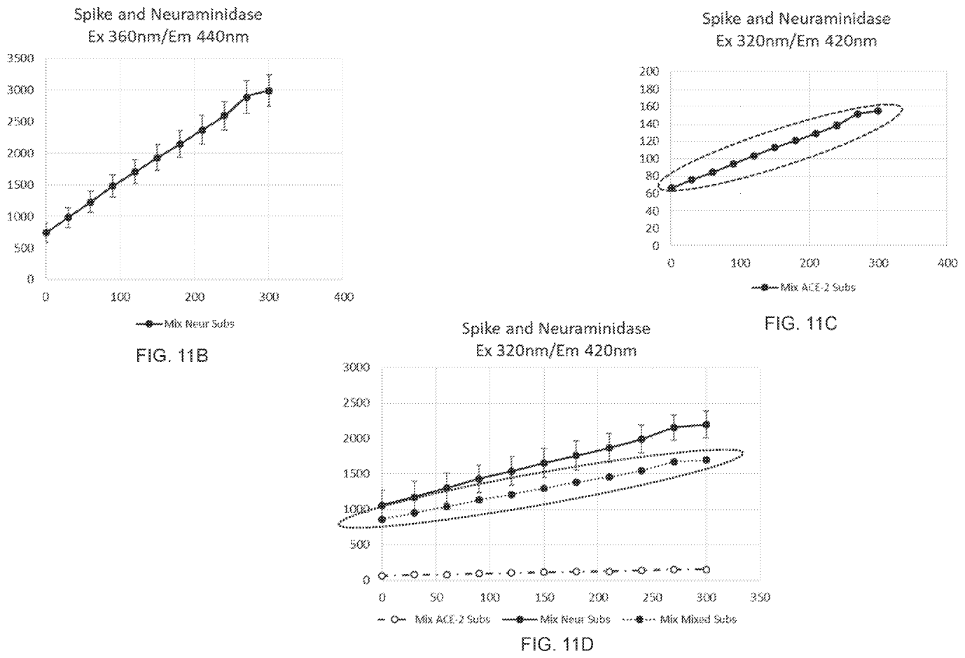

One envisioned form factor for the deployment of a viral detection technology is installed, continuous air monitoring devices that sample the air and run automated assays for virus detection in highly crowded areas, e.g. airports, stadiums, hospitals, schools, retail stores, and other buildings with a high flux of people. FLIR manufactures a bioaerosol trigger, the IBAC (Instantaneous Biological Aerosol Collector, FIG. 13) that detects biothreat particles in an airstream using a proprietary algorithm that analyzes particle size, count, and bio-fluorescence to classify biological threats. Once the trigger alarms, the IBAC can be coupled with a sampler that collects the particles for further analysis. The sensor is embedded in several DoD and DHS programs (CENTAUR, BD21) that seek to deploy real-time biosurveillance capabilities to upgrade our nation's ability to detect biological threats in the environment rapidly.

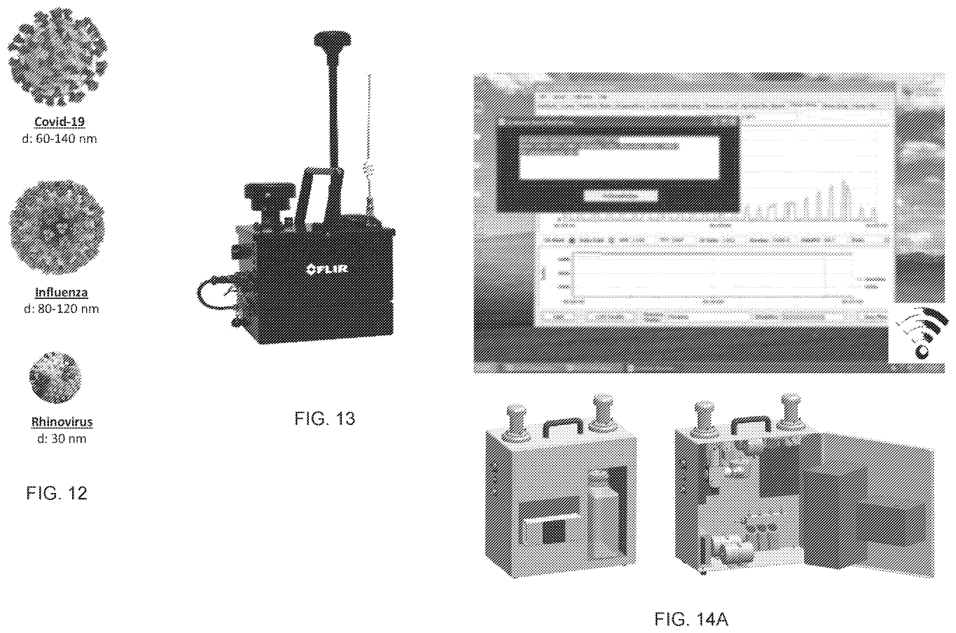

As part of a program to develop continuous sensors for nerve agent vapors, continuous enzymatic sensing chemistries have been previously developed by the Applicant that demonstrated detections as low as 0.25-1.4 μg/m³ of nerve agents, equating to sub-ppb detection limits, within two minutes—an unprecedented level of detection for these highly toxic chemical threats. The IBAC model and representative detection signal are shown in FIG. 14A and FIG. 14B. The present technology leverages this same approach and hardware to provide sensitive, real-time fluorescent assays that detect viral aerosols, enabling rapid, non-contact detection and identification.

### Example 2—SARS-Cov-2 Assay Chemistry

The inventors have leveraged unique surface protein properties and cellular entry mechanisms that viruses use to infect cells to develop highly specific assays that will be read by measurement of fluorescent signal development. The key innovations include virus-targeted, fluorescent, enzymatic chemistries that can be deployed for continuous airborne detection in the IBAC-Chem platform, or in a handheld test for exhaled breath for non-contact sampling and detection of the SARS-CoV-2 and Influenza viruses. What is particularly attractive is the built-in amplification capability that comes from exploiting the distinctive surface enzyme chemistries associated with pathogen binding and quantification.

As noted above, once bound by the Griffithsin, the cellular infection mechanism may be leveraged to identify the SARS-CoV-2 virus. Coronaviruses have surface-expressed S-proteins that serve as both the glycoproteins that are bound by the Griffithsin capture molecule, but which can also link a reporter enzyme that can be used to drive the detection response. Human angiotensin-converting enzyme 2 (ACE2) is a human cell surface-expressed enzyme that plays many roles in human physiology and is targeted by coronaviruses; this binding event then triggers the cellular entry that results in viral infection. SARS-CoV-2 has recently been found to target ACE2, similar to previous coronaviruses. However, it has an even stronger binding affinity than the SARS-CoV (K_{D}=1.2 nM for SARS-CoV-2 vs. 5.0 nM for SARS-CoV). This higher binding affinity is one possible explanation for why the novel coronavirus producing COVID-19 is more infectious than prior SARS and MERS coronavirus outbreaks that occurred throughout the early 2000s.

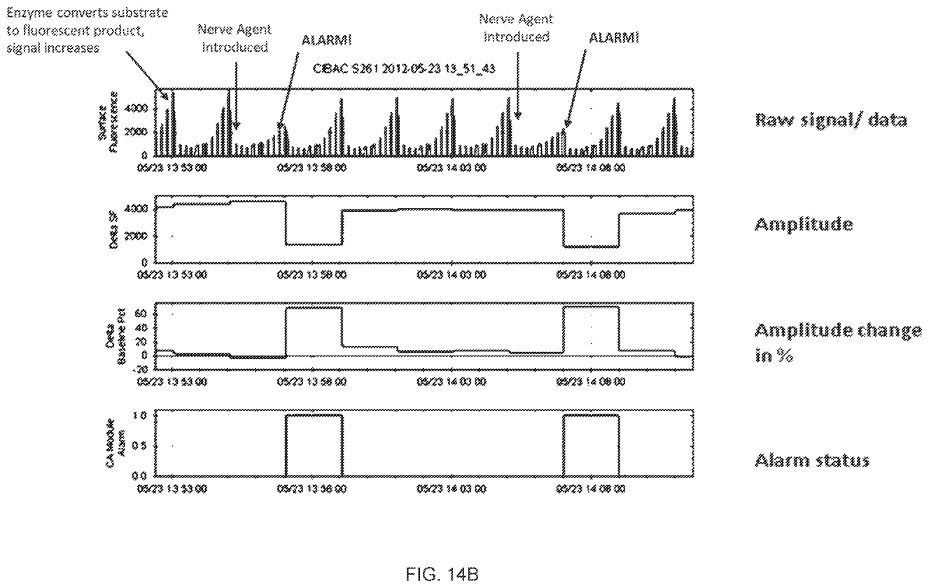

Upon viral capture, ACE2 enzyme may be nebulized from a liquid reservoir into the airstream, and then provide a buffer rinse to wash away any non-specifically bound ACE2. If a virus is present, ACE2 will bind to it and be retained. An ACE2 polypeptide-specific substrate, 7-methoxycoumarin-4-acetyl-alanine-proline-lysine-2,4-dinitrophenyl hydroxide, is then added. The 7-methoxycoumarin acetate (MCA) moiety is a fluorescent molecule, while the 2,4-dinitrophenol (DNP) moiety acts as a quencher. When covalently attached via a short Ala-Pro-Lys peptide sequence, DNP quenches fluorescence of MCA. If ACE2 is bound to the captured SARS-CoV-2 virus, ACE2 cleaves the peptide linkage, spatially separating the DNP quencher and permitting MCA fluorescence. This fluorescence can be quantitated at λₑₓ˜322 nm/λₑₘ˜381 nm in the near UV. A schematic of the assay and the fluorescence signal is depicted in FIG. 15.

### Example 3—Influenza Virus Assay Chemistry

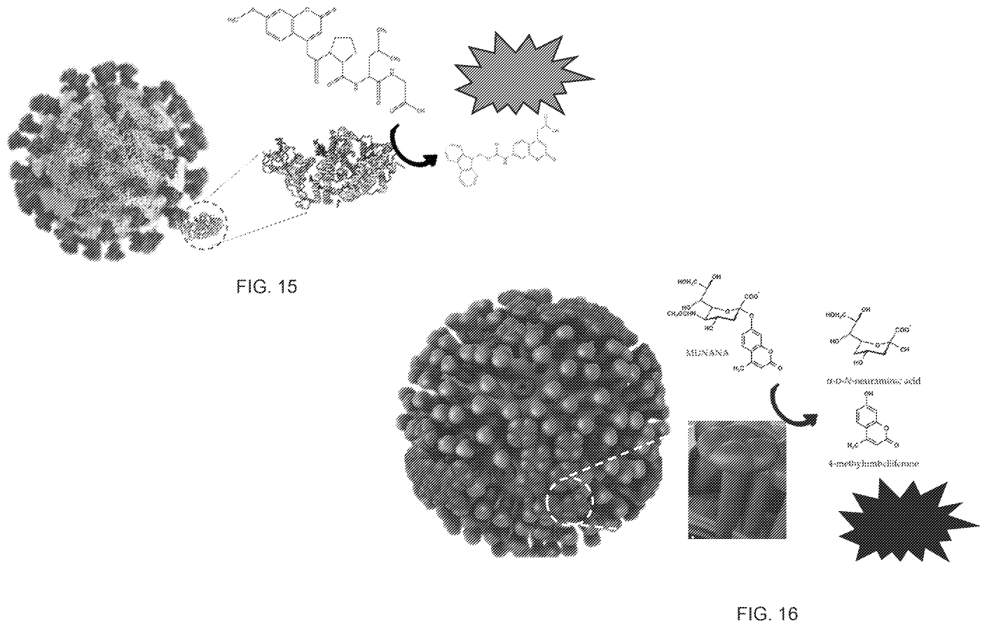

To detect influenza virus, its unique viral envelope protein features may be exploited. Flu virus contains surface hemagglutinin proteins that bind cell surface glycoproteins and facilitate cellular entry. Immobilized Griffithsin will capture the flu virus in the airstream via this hemagglutinin. Once captured, the enzymatic activity of another viral surface-bound protein, neuraminidase, can then be exploited. Neuraminidase is expressed on the viral envelope to cleave sialylated proteins in mucosal secretions and enable viral replicates to leave the cell. Neuraminidase hydrolyzes a synthetic substrate, MUNANA, producing neuraminic acid, and 4-methylumbelliferone (4-MU), which fluoresces at 440 nm (FIG. 16). This chemistry may be deployed to continuously monitor air for flu and to differentiate coronaviruses such as SARS-CoV-2.

Various aspects of certain non-limiting embodiments of inventions according to the present disclosure include, but are not limited to, the aspects listed in the following numbered clauses.
Clause 1. A method for detecting a virus in an airborne, aerosol, or aerosolized sample, comprising: contacting the sample with a biomolecule that specifically binds to, or is cleaved by, a surface protein or glycoprotein of the virus; and detecting the presence of the bound biomolecule with a detectable probe.
Clause 2. The method of clause 1, wherein the virus is a species in the genus Influenzavirus, Coronavirus, or Paramyxovirus.
Clause 3. The method of clause 1, wherein the viral-surface specific protein or glycoprotein is an ACE-2 protein, a hemagglutinin (HA) protein, a spike protein, or an F protein.
Clause 4. The method of clause 1, wherein the viral-surface specific protein is an enzymatically-active ACE-2 polypeptide.
Clause 5. The method of clause 2, wherein the virus is SARS-CoV-2, or a mutant or derivative thereof.
Clause 6. The method of clause 4, wherein the enzymatically-active ACE-2 polypeptide is detectable using a fluorescently-labeled substrate specific for an ACE-2 polypeptide thereof.
Clause 7. The method of clause 2, wherein the virus is rubella virus, morbillivirus, pneumovirus, or paramyxovirus.
Clause 8. The method of clause 2, wherein the Influenza virus is a human pathogenic serotype of Influenza A or Influenza B.
Clause 9. The method of clause 1, wherein the biomolecule is selected from the group consisting of a synthetic substrate, a self-quenched dye, a fluorescent dye, a fluorescent substrate, an electrochemiluminescent material, a labeled antibody, a labeled antigenic fragment, or any combination thereof.
Clause 10. The method of clause 9, wherein the synthetic substrate comprises MUNANA, which is hydrolyzable to neuraminic acid, and the detectable molecule, 4-methylumbelliferone (4-MU).
Clause 11. The method of clause 4, wherein the viral-surface specific protein comprises a neuraminidase polypeptide.
Clause 12. The method of clause 9, wherein the synthetic substrate comprises 7-methoxycoumarin 4-acetyl-alanine-proline-lysine-2,4-dinitrophenyl hydroxide.
Clause 13. The method of clause 12, wherein the synthetic substrate is cleaved to form the fluorescent label, 7-MCA, and the quencher, 2,4-dinitrophenol (DNP).
Clause 14. The method of clause 1, wherein the step of detecting is performed in near-real-time (i.e., on the order of one to three minutes).
Clause 15. A composition comprising: a) a lectin-based capture peptide or polypeptide; and b) an ACE-2 enzyme-specific fluorescent reporter system, adapted and configured for high-specificity detection and quantitation of a population of viral envelope glycoproteins.
Clause 16. The composition of clause 15, wherein the lectin-based capture peptide or polypeptide comprises a Griffithsin polypeptide or peptide, or an antibody or antigen-binding fragment that is specific for a Griffithsin polypeptide or peptide.
Clause 17. The composition of clause 16, wherein the lectin-based capture peptide or polypeptide comprises a Griffithsin polypeptide or peptide that comprises a sequence that is at least 98% identical to an at least 15, 20, or 25 contiguous amino acid sequence from SEQ ID NO:1.
Clause 18. The composition of clause 17, wherein the lectin-based capture peptide or polypeptide comprises a Griffithsin polypeptide or peptide that is at least 98% identical to an at least 15 contiguous amino acid sequence of SEQ ID NO:1.
Clause 19. The composition of clause 18, wherein the lectin-based capture polypeptide comprises the amino acid sequence of SEQ ID NO:1.
Clause 20. The composition of clause 15, wherein high-specificity detection and quantitation with a sampling rate of 4 L/min can yield about 40,000-fold amplifications/minute per viral particle within the population.
Clause 21. A viral detection assay system comprising the composition of clause 15.
Clause 22. A kit comprising: the viral detection assay system of clause 21, and instructions for using the system to detect a population of coronavirus or influenza virus particles in an airborne, aerosol, or aerosolized sample.
Clause 23. An article of manufacture, comprising: the viral detection assay system of clause 21, adapted and configured for use in a light scatter-based particle detector, such as a portable IBAC-2 device.
Clause 24. The article of manufacture of clause 23, further comprising a package insert having instructions for using said assay system to detect the presence of a coronavirus- or an influenza virus-specific peptide or polypeptide in a population of peptides or polypeptides obtained from an airborne environmental sample.
Clause 25. A system for quantitation of a population of viral particles in an airborne or aerosol sample in real-time, said system comprising; the article of manufacture of clause 23; and an IBAC-2 detector device operably configured to quantitate the population of viral particles in said system.
Clause 26. Use of a composition in accordance with clause 15, or a system in accordance with clause 25, in the detection of one or more viral particles in an airborne or aerosol sample.
Clause 27. Use in accordance with clause 26 in the near-real-time monitoring of viral particles in an air source such as an HVAC system.
Clause 28. A composition in accordance with clause 15, for use in detecting one or more coronaviral or influenza viral particles in an airborne or aerosol sample in real-time.
Clause 29. A method for detecting a virus, the method comprising:
   contacting an air sample including virus particles with a binding agent on a support, thereby binding at least a portion of the virus particles in the air sample to the binding agent on the support;
   contacting the virus particles bound to the binding agent on the support with a reporter that binds specifically to and/or is cleaved specifically by a surface protein of the virus particles or a glycoprotein of the virus particles; and
   detecting the presence of reporter bound to and/or cleaved by the surface protein of the virus particles or the glycoprotein of the virus particles.
Clause 30. The method of clause 29, wherein the air sample comprises the virus particles suspended in a gas.
Clause 31. The method of any one of clauses 29-30, wherein the virus particles in the air sample are aerosolized or airborne.
Clause 32. The method of any one of clauses 29-31, wherein the binding agent comprises at least one of a lectin, an antibody, and an antigen-binding fragment.
Clause 33. The method of any one of clauses 29-32, wherein the binding agent comprises a lectin and the lectin comprises a Griffithsin polypeptide or peptide.
Clause 34. The method of any one of clauses 29-33, wherein the binding agent comprises a lectin and the lectin comprises a Griffithsin polypeptide or peptide comprising an amino acid sequence that is at least 98% identical to a sequence of at least fifteen amino acids of SEQ ID NO:1.
Clause 35. The method of any one of clauses 29-34, wherein the support comprises at least one of a microplate and an impaction disk.
Clause 36. The method of any one of clauses 29-35, wherein the reporter comprises at least one of a substrate for an enzyme, a lectin bound to an enzyme, a labeled lectin, a labeled antibody, and a labeled antigenic fragment.
Clause 37. The method of any one of clauses 29-36, wherein the reporter comprises a substrate for an enzyme, the surface protein of the virus particles comprises the enzyme, and the detecting the presence of reporter bound to and/or cleaved by the surface protein of the virus particles or the glycoprotein of the virus particles comprises detecting the presence of a product formed from the substrate in the presence of the enzyme.
Clause 38. The method of any one of clauses 29-37, wherein the reporter comprises a substrate for an enzyme and the substrate comprises at least one of 4-methylumbelli-feryl N-acetyl α-D-neuraminic acid and 7-methoxycoumarin 4-acetyl-alanine-proline-lysine-2,4-dinitrophenyl hydroxide.
Clause 39. The method of clause 38, wherein the substrate is cleaved specifically by the surface protein of the virus particles or the glycoprotein of the virus particles to form 7-methoxycoumarin-4-acetic acid and 2,4-dinitrophenol.
Clause 40. The method of clause 36, wherein the labeled lectin, the labeled antibody, and/or the labeled antigenic fragment comprises at least one of a dye and an enzyme bound to the respective lectin, antibody, and/or antigenic fragment.
Clause 41. The method of clause 40, wherein the labeled lectin, the labeled antibody, and/or the labeled antigenic fragment comprises at least the dye, and wherein the dye is a self-quenched dye, a fluorescent dye, or an electrochemiluminescent dye.
Clause 42. The method of clause 41, wherein detecting the presence of reporter bound to and/or cleaved by the surface protein of the virus particles or the glycoprotein of the virus particles comprises exciting the dye and detecting an emission of the dye.
Clause 43. The method of clause 36, wherein the reporter comprises the lectin bound to the enzyme, the method further comprises contacting the reporter with a substrate for the enzyme, and wherein detecting the presence of reporter bound to and/or cleaved by the surface protein of the virus particles or the glycoprotein of the virus comprises detecting a product formed from the substrate in the presence of the enzyme.
Clause 44. The method of any one of clauses 29-43, wherein the virus particles comprise at least one species in at least one genus of Influenzavirus, Coronavirus, or Paramyxovirus.
Clause 45. The method of any one of clauses 29-44, wherein the virus particles comprises SARS-CoV-2, a mutant thereof, or a derivative thereof.
Clause 46. The method of any one of clauses 29-44, wherein the virus particles comprise at least one of rubella virus, morbillivirus, pneumovirus, paramyxovirus, a human pathogenic serotype of Influenza A, and a human pathogenic serotype of Influenza B.
Clause 47. The method of any one of clauses 29-46, wherein the surface protein of the virus particles or the glycoprotein of the virus particles comprises at least one of an angiotensin-converting enzyme 2 (ACE-2) protein, a hemagglutinin protein, a spike protein, a neuraminidase polypeptide, and an F protein.
Clause 48. The method of any one of clauses 29-47, wherein the surface protein of the virus particles or the glycoprotein of the virus particles comprises an angiotensin-converting enzyme 2 (ACE-2) protein.
Clause 49. The method of any one of clauses 29-46, wherein detecting the presence of reporter bound to and/or cleaved by the surface protein of the virus particles or the glycoprotein of the virus particles is performed in no greater than sixty minutes.
Clause 50. A system for detecting the presence of virus particles, the system comprising:
   a support comprising a binding agent configured to bind at least a portion of virus particles in an air sample;
   an air pump configured to deliver the air sample to the support;
   a fluid delivery apparatus configured to deliver a reporter to the support, wherein the reporter is configured to bind specifically to and/or to be cleaved specifically by a surface protein of the virus particles or a glycoprotein of the virus particles;
   a sensor; and
   a programmable hardware device configured to measure an electromagnetic property of the support utilizing the sensor to thereby determine presence of the reporter.
Clause 51. The system of clause 50, further comprising a light scatter-based particle detector configured to determine a quantity of particles in the air sample.
Clause 52. The system of any of clauses 50-51, wherein the air sample is from a heating, ventilation and air-conditioning system.
Clause 53. The system of any of clauses 50-52, wherein the system is configured to perform a cycle including cyclically delivering the sample to the support, delivering the reporter to the support, and measuring the property of the support.
Clause 54. The system of clause 53, wherein the cycle is no longer than sixty minutes.

### REFERENCES

The following references, to the extent that they provide exemplary procedural or other details supplementary to those set forth herein, are specifically incorporated herein in their entirety by express reference thereto:
PCT Intl. Pat. Appl. Publ. No. WO 2013/088367.
BALZARINI, J, “Targeting the glycans of glycoproteins: a novel paradigm for antiviral therapy,” Nat. Rev. Microbiol., 5(8):583-597 (2007).
BIUSO, F et al., “Use of lentiviral pseudotypes as an alternative to reassortant or Triton X-100-treated wild-type Influenza viruses in the neuraminidase inhibition enzyme-linked lectin assay,” Influenza Other Respi. Viruses, 13:504-516 (2019).
COUZENS, L et al., “An optimized enzyme-linked lectin assay to measure influenza A virus neuraminidase inhibition antibody titers in human sera,” J. Virol. Methods, 210:7-14 (2014).
DAN, X et al., “Development and Applications of lectins as biological tools in biomedical research,” Medicin. Res. Rev., 36(2):221-247 (2016).
GAO, J et al., “Measuring influenza neuraminidase inhibition antibody titers by enzyme-linked lectin assay,” J. Vis. Exp., 115:54573 (2016).
LUSVARGHI, S and BEWLEY, CA, “Griffithsin: an antiviral lectin with outstanding therapeutic potential. Viruses, 2016, 8:296 (2016).
M3TD MAC Chem. Technical Assessment Test Report (2012).
MCCOY, Jr., J P et al., “Enzyme-linked lectin assay (ELLA): II. Detection of carbohydrate groups on the surface of unfixed cells,” Exp. Cell Res., 151:96-103 (1984).
PREVATO, M et al., “An innovative pseudotypes-based enzyme-linked lectin assay for the measurement of functional anti-neuraminidase antibodies,” PLoS ONE, 10(8): e0135383 (2015).
SLIFKIN, M, and RJ DOYLE, “Lectins and their application to clinical microbiology.” Clin. Microbiol. Rev., 3(3):197-218 (1990).
SUDA, Y et al., “Highly sensitive detection of influenza virus in saliva by real-time PCR method using sugar chain-immobilized gold nanoparticles; application to clinical studies,” Biotechnol. Rep. (Amsterdam), 7:64-71 (May 2015).
VAHEY, M D et al., In: Chakraborty, AK (ed), Neher, RA (ed). Influenza A virus surface proteins are organized to help penetrate host mucus, eLife, 8: e43764 (2019).
VAN BREEDAM et al., “Bitter-sweet symphony: glycan-lectin interactions in virus biology,” FEMS Microbiol. Rev., 38(4):598-632 (July 2014).
WALLS, A C et al., “Structure, function, and antigenicity of the SARS-CoV-2 spike glycoprotein,” Cell, 180:1-12 (2020).

It should be understood that the examples and embodiments described herein are for illustrative purposes only and that various modifications or changes in light thereof will be suggested to persons skilled in the art and are to be included within the spirit and purview of this application and the scope of the appended claims. All references, including publications, patent applications and patents, cited herein are hereby incorporated by reference to the same extent as if each reference was individually and specifically indicated to be incorporated by reference and was set forth in its entirety herein. Recitation of ranges of values herein are merely intended to serve as a shorthand method of referring individually to each separate value falling within the range, unless otherwise indicated herein, and each separate value is incorporated into the specification as if it were individually recited herein.

The description herein of any aspect or embodiment of the invention using terms such as “comprising,” “having,” “including,” or “containing” with reference to an element or elements is intended to provide support for a similar aspect or embodiment of the invention that “consists of,” “consists essentially of,” or “substantially comprises” that particular element or elements, unless otherwise stated or clearly contradicted by context (e.g., a composition described herein as comprising a particular element should be understood as also describing a composition consisting of that element, unless otherwise stated or clearly contradicted by context).

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents that are chemically and/or physiologically related may be substituted for the agents described herein while the same or similar results would be achieved.

1. A method for detecting a virus, the method comprising:
contacting an air sample including virus particles with a binding agent on a support, thereby binding at least a portion of the virus particles in the air sample to the binding agent on the support;
contacting the virus particles bound to the binding agent on the support with a reporter that binds specifically to and/or is cleaved specifically by a surface protein of the virus particles or a glycoprotein of the virus particles; and
detecting the presence of the reporter bound to and/or cleaved by the surface protein of the virus particles or the glycoprotein of the virus particles.

2. The method of claim 1, wherein the air sample comprises the virus particles suspended in a gas.

3. The method of claim 1, wherein the virus particles in the air sample are aerosolized or airborne.

4. The method of claim 1, wherein the binding agent comprises at least one of a lectin, an antibody, and an antigen-binding fragment.

5. The method of claim 1, wherein the binding agent comprises a lectin and the lectin comprises a Griffithsin polypeptide or peptide.

6. The method of claim 1, wherein the binding agent comprises a lectin and the lectin comprises a Griffithsin polypeptide or peptide comprising an amino acid sequence that is at least 98% identical to a sequence of at least fifteen amino acids of SEQ ID NO: 1.

7. The method of claim 1, wherein the support comprises at least one of a microplate and an impaction disk.

8. The method of claim 1, wherein the reporter comprises at least one of a substrate for an enzyme, a lectin bound to an enzyme, a labeled lectin, a labeled antibody, and a labeled antigenic fragment.

9. The method of claim 1, wherein the reporter comprises a substrate for an enzyme, the surface protein of the virus particles comprises the enzyme, and the detecting the presence of reporter bound to and/or cleaved by the surface protein of the virus particles or the glycoprotein of the virus particles comprises detecting the presence of a product formed from the substrate in the presence of the enzyme.

10. The method of claim 1, wherein the reporter comprises a substrate for an enzyme and the substrate comprises at least one of 4-methylumbelli-feryl N-acetyl α-D-neuraminic acid and 7-methoxycoumarin 4-acetyl-alanine-proline-lysine-2,4-dinitrophenyl hydroxide.

11. The method of claim 10, wherein the substrate is cleaved specifically by the surface protein of the virus particles or the glycoprotein of the virus particles to form 7-methoxycoumarin-4-acetic acid and 2,4-dinitrophenol.

12. The method of claim 8, wherein the labeled lectin, the labeled antibody, and/or the labeled antigenic fragment comprises at least one of a dye and an enzyme bound to the respective lectin, antibody, and/or antigenic fragment.

13. The method of claim 12, wherein the labeled lectin, the labeled antibody, and/or the labeled antigenic fragment comprises at least the dye, and wherein the dye is a self-quenched dye, a fluorescent dye, or an electrochemiluminescent dye.

14. The method of claim 13, wherein detecting the presence of the reporter bound to and/or cleaved by the surface protein of the virus particles or the glycoprotein of the virus particles comprises exciting the dye and detecting an emission of the dye.

15. The method of claim 8, wherein the reporter comprises the lectin bound to the enzyme, the method further comprises contacting the reporter with a substrate for the enzyme, and wherein detecting the presence of reporter bound to and/or cleaved by the surface protein of the virus particles or the glycoprotein of the virus comprises detecting a product formed from the substrate in the presence of the enzyme.

16. The method of claim 1, wherein the virus particles comprise at least one species in at least one genus of Influenzavirus, Coronavirus, or Paramyxovirus.

17. The method of claim 1, wherein the virus particles comprises SARS-CoV-2, a mutant thereof, or a derivative thereof.

18. The method of claim 1, wherein the virus particles comprise at least one of rubella virus, morbillivirus, pneumovirus, paramyxovirus, a human pathogenic serotype of Influenza A, and a human pathogenic serotype of Influenza B.

19. The method of claim 1, wherein the surface protein of the virus particles or the glycoprotein of the virus particles comprises at least one of an angiotensin-converting enzyme 2 (ACE-2) protein, a hemagglutinin protein, a spike protein, a neuraminidase polypeptide, and an F protein.

20. (canceled)

21. (canceled)

22. A system for detecting the presence of virus particles, the system comprising:
a support comprising a binding agent configured to bind at least a portion of virus particles in an air sample;
an air pump configured to deliver the air sample to the support;
a fluid delivery apparatus configured to deliver a reporter to the support, wherein the reporter is configured to bind specifically to and/or to be cleaved specifically by a surface protein of the virus particles or a glycoprotein of the virus particles;
a sensor; and
a programmable hardware device configured to measure an electromagnetic property of the support utilizing the sensor to thereby determine presence of the reporter.

23. (canceled)

24. (canceled)

25. (canceled)

26. (canceled)
